SEQUENCE LISTING (32 sequences)

SEQ ID NO: 1 - DNA, 74997 bp, synthetic construct
  misc_feature 1..74997: note = Synthetic
  source 1..74997: mol_type = other DNA
ccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggcctcgaggcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaactcaacgtaaaaacaacttcagacaatacaaatcagcgacactgaatacggggcaacctcatgtcccccccccccccccccctgcaggcatcgcggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcgtgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtccagaggtccgattccggctaactaactagcttaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgaaaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaaggcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggattcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcacaccggttaaaaccaaaatccagtggcgagctctcgagtcgatcgctatcaactttgtatagaaaagttgggccgaattcgagctcggtacggccagaatggcccggaccgggttaccgaattcgagctcggtaccctgggatcagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatttcgaattttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatgtcgacgggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgttcgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcggtcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatggcagagccgaacaagggtggagcacctgcgatgaagaacgtcgccaagccgtcgaccaaacgcctgatcccgagctcgatagccgcttcgagccagactagcgccaacgctctgacggagccccttcctgggtctgacgcgatcggccagagctacgacgcattcgggttcttcgccaatccccgcagcatcatgaaggagctgttcgagttcagcccacaggaggagatcgtcgtcgaaggcaacacctggcttctcagcagcgacttcgtctacaccgccatccgcgacacagagacctcgaccgtctcgaggcgcaccaaggacgactacagcaaggagctggccgtgaaggtgaagctcagcggaagctacggctacttcagcgctagcgtggagagcgacttcagccagagcatcagcgacgctaccgacacgacgtacaccagcgtccgcacccacgtcaacaagtggcgcctcagcctcaaggacgacgtcggagctctccggagcaagctccttcctggtgtcaagcaggctctcgctacgatggacgcaacccagctcttcgacacgttcggcacccactacgtcagcgaggtcctcgtcggtggaagagccgactacgtcgccaccaccaagaccagcgccttcagctcgagcacctccatcagcgtcgctgcagaggcgtcgtttcagagcatcgcaggaggcgaagtcagccccgagagcaaggtcctcgccgagatgctgcgcgagaactccagcacacggctctacgcactgggaggctcagcactcccgaacatcacggacccagcgacctacaacgcctggctggagagcatcgacaccatcccggtcttctgcggcttcacccagaactccctcaagtcgatcagcgagctcgcggattcagcccaacgcagagacgcactcgcgaaggcatcccagtcgtacatccccagctacgtcactcgcccagcagtcgtgggcctcgaggtcatcatctccgactccaactccgagagccctccatacggctacacccggatcgactacgacctcaaccgcaatgccggaggcaagtacgtcttcctctgctacaagcagaagaacatctccgtcggaggtgacgcagacgcgatcacggacgtcctcgtcgtctacggcaacgaccggaacccaagcgtgccctcaggctacaccaagatcgacaaggacctgaactccggagctggagggaagtacatctacttctgctactccaaggacaagcgcaagcaggaggagggccttccgatacgcggacttcgcgtcgttggaccacaccctacgtcagtggcaccgtacggcttcagcaagatcgacatcgacctcaacatgggcgcaggtggggacttcatctacctctgcaagtcgcggcacctcgagtgagttaaccccgggtcaacccatcaggaaggatgaagcgcccctcattttgtgccctaggtcgtggattgctggattttaattttacacatttccttgtcgatcctttctgctgtgtgtggttcgagaatgttagtgtgttatcgcaagatctgggtgtttggaagttatctcattattgggcctcataaattcataattcttgccagttagtgacaactgtagcttaggtttacttctgcttgtagtacatcgcctagatcgtgggagtccctcttttcagacgaatgtcatgaaacattggtttttggaaatgattaggaagacatttgctgttttgtcgactgctgttttttacggccaagttcagagttttttttttcatgtacaaagtatcagcagttaaattatgttacctttaccatggttcttcatatttgttttccttccattgctcaatctatgtcatcttttgaaatggtttggaaggcatcctttataggatatatagatatagatttgaagcataattgttaggataagacaccagctagcctatgctgcaacgcacattattcgacccttaataacaacaggtgatttttatattataaaaaagttggaaaagtatacacaagaatttttcaaaagaagggtaaaagggaacagccctcctgctcgacaattggaattggtgtcccgcataatttttttctgcctttgagaattcaggcgtctctggattctagttcaccatttaccaattagaaggaatactatgtatgtataattctacaatctgcattctacacaatccttctattttctgaatatagttgcaagactagggctctcttatagtatttctaattatagccgctttgcaaagtactgtcatatttgattaggggtattggaaagaaggagaaaagggtgacaccctgcttgacaattggaattgataagcagccagggtaccaaggcgcgaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccattctatcactagctagctgctaattattcccgggcacccagctttcttgtacaaagtggccgttacagaatcactgactagctaatctagcggccgctcaagcttcggcatgcaattcgcatacctacagtacaacgtggccaaagtcatcatttaatgagctctcgggcgcgccgtctcactagctagctgctaacgttcccgggcaactttattatacaaagttgatagatcctacaggccagaatggcctctggattcagcggcctagaaggccgaagtacttggtcttcctaatatcggaccgaggaccgattaaactttaattcggtccgtcaatattcaccgaagcgactaattaactagctgtcccacggcctaactagcacttaatcccctagcctaacctaagagcgctaatctaggctagtggtcacttagggctttaaggctagcgtatacgaagttcctattccgaagttcctattcttcaaaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggggcgcgccaataccgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacg

SEQ ID NO: 2 - DNA, 24867 bp, synthetic construct
  misc_feature 1..24867: note = Synthetic
  source 1..24867: mol_type = other DNA
cgtactgcactgccaccattcaagacaagctatggtcgtcgccgtgaacgtgagccgcgcatcgttgaaggctaggtccacgaggaggtagacaagggcacatgcgctcccaagagcttgttcgtgttcgtgacccgacgaccgcgcgcgtttgtccgtgggctcgtgggacgccagcagcccacgcgaaacggttcccctgccgcgcgcgcgcgcgcccggtgcatggctctcctcagcctccgaatggagtcatcaccgtcgacgcctcgacggcgcggaggtgaccaaataaggtccggctgctcgcagccgtggcacgaaacgaggacacacggactggtaggaggctagggggggcacggacaagaccgctccgttccgttgcctctcccgcgctctgcgagggatgatgcatgggcagcgcgcggacacacggactggcagcggccctgtacccacgtcgcagtgccgggatccgcgcctaccacggcacgatcagcgtcatcatggcacaatcattcacgaacctcccagtcccagcggattgcacgccgaaaaatcgtcctctagaagaaagctttttctcgtcgcacatcggcgcttctgctcggcacggcatgtgaaactgtcagggcacaggggtaggtttactgacaaggtttgatttcttttccggctgctgagcacggtccgcgactgggagcaaattcaatgcccttgctttctcccgtcccggcccattcaaaccgggccgtcgttttagtttctttttatgttttacttggttttagcggcgaggcgatgcaacgaagaccacagcagcaaactactattgtttcagggtcatttctaatacactctccgtcctaaaatattaggtggcgcttggcagagtttccacagcttcgctcagagcgagaatcactcttactgagctaaatgataaaaatcggaactgcttcatgttgagagtggagtgaatctagtctacactatgtattagaaagtgagagaaaaaaaacatgcttcccactacactactcccaacatgctccccactctttaacccctcatgaatcatttcacagcctatttgccaaacaattttcgtcaaatatattcacttctagtagagaatcactcagaatcaccaatcagctctcggaactagagaattgatcaaagacaaactctattaccttcaatgatatgtggttatgattcatgttggcaagctgtgatcctgttgctgctgttcccctggtacttttttttttgtgtgccggttgagtttttatagtttatgttgtgctgcatatgaatgagttttggtagttaccgtggttgccgttcacagcggcgttgcctgctgttcatatgttggcaatccgtgaatttgcatgcttctatatatgagtttggtgttcaattgtacttatatactaccaaaaaataccatagtttcagtgggctgaagaaattaaaacccatgcatgctagtaatgtcatagctaggtgtataccatggttttccaataccaaacttagctaccaaacacatactactgcatctgtatatataatgcactggtgtgcagccgtcagccgtgtgccttctgaagcgtgaaacctggtaaaaaaaaaaaacgggggaggcaagaagcatcagggcaggcaggaggatcctagtacatatcctacttactggcttgcagcaaggctagctgatggctgcgtgcgtgcttacatgatgatgatcatgcgtgtcgttcgtactcgtacgaggatcgaggaaagacaccataactcaccttcaacagacacccttcgttaggagcatgcacggatagatggcgtctagcatatcgataggacatgacaagtggtacgatccccgtcacatgtccatggaggcatctgatatggacacggcgtgtatctatcgcggctggaacagaaccagcgctcgcgcggcggtcggcgggagggacagaccttggctccgtgcgttcaggttgtgcttgtgccgcgcgccacgcacggtctccgccgcctgcagctgaaattttagatttacatcctatccctttattttttttatttgtcacaattcagttcaaaaatgaagaacggaggtagtgcatcctttgtgagactaatgaaaatcacatctggatcctgaaatcggcgtcgtaacctacaaggccacggactggattagatagtggtccatggtgcataatgaggatcgaggggtacctcactgactagctaatcgagctagttaccctatgaggtgacatgaagcgctcacggttactatgacggttagcttcacgactgttggtggcagtagcgtacgacttagctatagttccggacttaccgataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataacataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatttcgaattttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatgtcgacgggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgttcgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcggtcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatggcagagccgaacaagggtggagcacctgcgatgaagaacgtcgccaagccgtcgaccaaacgcctgatcccgagctcgatagccgcttcgagccagactagcgccaacgctctgacggagccccttcctgggtctgacgcgatcggccagagctacgacgcattcgggttcttcgccaatccccgcagcatcatgaaggagctgttcgagttcagcccacaggaggagatcgtcgtcgaaggcaacacctggcttctcagcagcgacttcgtctacaccgccatccgcgacacagagacctcgaccgtctcgaggcgcaccaaggacgactacagcaaggagctggccgtgaaggtgaagctcagcggaagctacggctacttcagcgctagcgtggagagcgacttcagccagagcatcagcgacgctaccgacacgacgtacaccagcgtccgcacccacgtcaacaagtggcgcctcagcctcaaggacgacgtcggagctctccggagcaagctccttcctggtgtcaagcaggctctcgctacgatggacgcaacccagctcttcgacacgttcggcacccactacgtcagcgaggtcctcgtcggtggaagagccgactacgtcgccaccaccaagaccagcgccttcagctcgagcacctccatcagcgtcgctgcagaggcgtcgtttcagagcatcgcaggaggcgaagtcagccccgagagcaaggtcctcgccgagatgctgcgcgagaactccagcacacggctctacgcactgggaggctcagcactcccgaacatcacggacccagcgacctacaacgcctggctggagagcatcgacaccatcccggtcttctgcggcttcacccagaactccctcaagtcgatcagcgagctcgcggattcagcccaacgcagagacgcactcgcgaaggcatcccagtcgtacatccccagctacgtcactcgcccagcagtcgtgggcctcgaggtcatcatctccgactccaactccgagagccctccatacggctacacccggatcgactacgacctcaaccgcaatgccggaggcaagtacgtcttcctctgctacaagcagaagaacatctccgtcggaggtgacgcagacgcgatcacggacgtcctcgtcgtctacggcaacgaccggaacccaagcgtgccctcaggctacaccaagatcgacaaggacctgaactccggagctggagggaagtacatctacttctgctactccaaggacaagcgcaagcaggaggagggccttccgatacgcggacttcgcgtcgttggaccacaccctacgtcagtggcaccgtacggcttcagcaagatcgacatcgacctcaacatgggcgcaggtggggacttcatctacctctgcaagtcgcggcacctcgagtgagttaaccccgggtcaacccatcaggaaggatgaagcgcccctcattttgtgccctaggtcgtggattgctggattttaattttacacatttccttgtcgatcctttctgctgtgtgtggttcgagaatgttagtgtgttatcgcaagatctgggtgtttggaagttatctcattattgggcctcataaattcataattcttgccagttagtgacaactgtagcttaggtttacttctgcttgtagtacatcgcctagatcgtgggagtccctcttttcagacgaatgtcatgaaacattggtttttggaaatgattaggaagacatttgctgttttgtcgactgctgttttttacggccaagttcagagttttttttttcatgtacaaagtatcagcagttaaattatgttacctttaccatggttcttcatatttgttttccttccattgctcaatctatgtcatcttttgaaatggtttggaaggcatcctttataggatatatagatatagatttgaagcataattgttaggataagacaccagctagcctatgctgcaacgcacattattcgacccttaataacaacaggtgatttttatattataaaaaagttggaaaagtatacacaagaatttttcaaaagaagggtaaaagggaacagccctcctgctcgacaattggaattggtgtcccgcataatttttttctgcctttgagaattcaggcgtctctggattctagttcaccatttaccaattagaaggaatactatgtatgtataattctacaatctgcattctacacaatccttctattttctgaatatagttgcaagactagggctctcttatagtatttctaattatagccgctttgcaaagtactgtcatatttgattaggggtattggaaagaaggagaaaagggtgacaccctgcttgacaattggaattgataagcagccagggtaccaaggcgcgaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccattctatcactagctagctgctaattattcccgggcacccagctttcttgtacaaagtggccgttacagaatcactgactagctaatctagcggccgctcaagcttcggcatgcaattcgcatacctacagtacaacgtggccaaagtcatcatttaatgagctctcgggcgcgccgtctcactagctagctgctaacgttcccgggcaactttattatacaaagttgatagatcctacaggccagaatggcctctggattcagcggcctagaaggccgaagtacttggtcttcctaatatcggaccgaggaccgattaaactttaattcggtccgtcaatattcaccgaagcgactaattaactagctgtcccacggcctaactagcacttaatcccctagcctaacctaagagcgctaatctaggctagtggtcacttagggctttaaggctagcgtatacgaagttcctattccgaagttcctattcttcaaaaagtataggaacttctgtacacctgagctgattccgatgacttcgtaggttcctagctcaagccgctcgtgtccaagcgtcacttacgattagctaatgattacggcatctaggaccgactagctaactaactagggcgcgccatgaggagcaatcattgttcaagacatgatgcaaagctagaaaactttgattgtggccgtcctaattgtgaagtttaggccggggggaacttcatgaaccctatcgaagcttaattagttcttttttgttgttagccatgtttgtattgtagtttaggtgaacaacatgacgccgcacccgcgatctcagggctcgtccccacacaggagggcacgtcgtcgtcttcgccgccgagcatcagagattcagagcacgtacacgcacatctcaagcaaacggagtagtacgtcctactcctacgtacatacctagccgacgacctttatgtgcacaccaccactgctctgctgcccggcctctccgtcgtccgttcatcaccagctggtctggtccttcaatttccatgcgtcggtccgaagtcaacattttccgtcaattcatggccaaaggctacaactaacattgtttaatgtcgactgttttttttttgttaatgtgacgccgtgttttttttgttattgctagaacactgtttaatgtcaaactgttttaagtccatgggacgcctcagcaatagtagccttttgttaatgtgacgccgcatcaatcaactaattaaagcccttaaccatctgtgtctcattgttaccggcatcacctaaacaacagatcacggctctacgagcaacgtacataacagtaaactaatggcctgatctgctgggctagtagggccacagcaacactagctgaacgtatgcagcggcggcggcggcaccatccaagtaactcaagcgagcacggttgaacaatacttgaacctttttgtcccaatccactttagaaaactaagaaattcatatcggagaaaggtcgaagaatggtttgagaaaagactcggtagcttccccaccaagtctcacaatagaaacattatgaaaatatttacccttcataaccctaatatgacccttcttcgcagtcgacttctcaaagacgacgtggcttggtttctagggtcgttcttcggcatcaccctcaaaatcaaagtcctcgctatcctcagaatcgttgacattccgaccaatattttataagtaatcttcggaatattactcttctccatagcctcctggaacccagcaagcgcaaactcttcactcttgctcccctcgaccatcatagccaaaaccaaataaccaatgaatctaaaacggtcacaattgataataataaccttcggttttaaagcgctagctattctagcaacaaaataggtgaatggtgtgataattgcgcatacgattttcattctatttataataaaagccaggccagggtcgaataaaaaatataaaactaccactgcaaaatgggcctcgcatttcaagtatctcgatgattagcttaaggaaggtgttttttcaaccttcgacataaggccttcgttcatttctcagtttgagttcgttgcaagaaaacaaactaatactgggaggggctagttttggggccttcatcatttgaaggttctcaaaacactaattaaccattgtttcatgatacatctctaaaagtgttacaggtgcttcagtaaaaaccaccttcgaaataaatcgagcaacgacgaaggtacatttctactagcggataaaactggtcttgggccgaggcagcatacaaagaagcttcgcctgtgcgcgcaagtggcagaggtgttagccgaagtcgataactttggcaacaacagaagaggatggagaaaatacattattatcctgatggtatttgtaaacaatgtttgtaaaaccttggggcatgattataattctatataaagacagttcattttcctataaataggtgagcagtaccctgcataaggcatctttttgaggggtgatcacttcgtttaacttagcttacagagaacctttgtaccatcttgtgtgtggagctgaaggtatgcttgtacacctttatcaagagagatagagtaaattgctaaggcatataagatgagttgaatatgaagttacactttg

SEQ ID NO: 3 - DNA, 24867 bp, synthetic construct; identical to SEQ ID NO: 2
  misc_feature 1..24867: note = Synthetic
  source 1..24867: mol_type = other DNA

SEQ ID NO: 4 - DNA, 1434 bp, Ophioglossum pendulum
  source 1..1434: mol_type = other DNA
atggaacccaacaaaggtggtgctccagccatgaaaaatgtagccaaaccttccacaaagagactcataccgtcatccattgctgcatcatcgcaaacatctgcaaatgcattaaccgagccattgcctggctctgatgccatcggccagagctacgatgcattcgggttcttcgccaatcccagaagcatcatgaaggagttgttcgagttcagtcctcaagaggagatagtcgtggaaggaaatacctggcttctctcttcggacttcgtttacaccgcaattagagacactgagacctccacggtctcacgtcgcaccaaggacgactactccaaggaattggctgtgaaggtgaagctgtctggaagctatggctatttttctgcctccgttgagtctgatttctctcagagcatttccgatgcgacggacaccacatacacttcagttcgcacccacgtcaacaagtggaggctgagcctgaaggacgatgttggggcccttcgctcgaagctgctgcctggcgtcaagcaggcgctggctaccatggatgctacgcagctctttgacacgttcgggactcactatgtgagcgaggtgcttgtgggtgggcgtgcagactacgttgccaccaccaagaccagcgcgttcagctcatccaccagcatcagcgtagcggcagaagcatcattccagtccatcgcgggtggggaggtgtctccggagtccaaggtgcttgcggagatgctgagagagaacagcagcacacgcctgtacgctcttggcgggagtgccctccccaacatcacagatccagccacgtacaacgcctggctggagtccattgacaccatccctgtcttctgcggattcactcaaaacagcctcaagtccatctcggagcttgctgactccgctcaacgccgggacgctctggccaaagcctcccaaagctatatcccatcatacgtgactcgtcctgcagtggttggcttggaggtcataatatcggacagcaactcggagagccctccctatggctacaccagaatagactacgacctcaaccgcaacgcaggaggcaaatacgtgttcctctgctacaagcagaagaacatttcagtgggaggggatgccgatgccatcaccgacgtccttgtcgtctacggaaatgaccgaaatccatctgtgccgtctggctacaccaagatcgacaaggacctcaactccggggcaggcggtaagtatatctacttctgctactccaaggacaagcggaagcaagaggagggactgcccattcgtgggcttcgtgttgttgggcctcacccaacctcagtggcaccttacggattctctaagatcgacatagatctgaatatgggtgcaggaggagattttatatacctttgcaaatcacgccatttggag

SEQ ID NO: 5 - protein, 478 aa, Ophioglossum pendulum
MEPNKGGAPAMKNVAKPSTKRLIPSSIAASSQTSANALTEPLPGSDAIGQSYDAFGFFANPRSIMKELFEFSPQEEIVVEGNTWLLSSDFVYTAIRDTETSTVSRRTKDDYSKELAVKVKLSGSYGYFSASVESDFSQSISDATDTTYTSVRTHVNKWRLSLKDDVGALRSKLLPGVKQALATMDATQLFDTFGTHYVSEVLVGGRADYVATTKTSAFSSSTSISVAAEASFQSIAGGEVSPESKVLAEMLRENSSTRLYALGGSALPNITDPATYNAWLESIDTIPVFCGFTQNSLKSISELADSAQRRDALAKASQSYIPSYVTRPAVVGLEVIISDSNSESPPYGYTRIDYDLNRNAGGKYVFLCYKQKNISVGGDADAITDVLVVYGNDRNPSVPSGYTKIDKDLNSGAGGKYIYFCYSKDKRKQEEGLPIRGLRVVGPHPTSVAPYGFSKIDIDLNMGAGGDFIYLCKSRHLE

SEQ ID NO: 6 - DNA, 27 bp, synthetic construct
  misc_feature 1..27: note = Synthetic
  source 1..27: mol_type = other DNA
gcatctaggaccgactagctaactaac

SEQ ID NO: 7 - DNA, 24 bp, synthetic construct
  misc_feature 1..24: note = Synthetic
  source 1..24: mol_type = other DNA
ctttgcatcatgtcttgaacaatg

SEQ ID NO: 8 - DNA, 15 bp, synthetic construct
  misc_feature 1..15: note = Synthetic
  source 1..15: mol_type = other DNA
cgccatgaggagcaa

SEQ ID NO: 9 - DNA, 18 bp, synthetic construct
  misc_feature 1..18: note = Synthetic
  source 1..18: mol_type = other DNA
gctggccgtgaaggtgaa

SEQ ID NO: 10 - DNA, 20 bp, synthetic construct
  misc_feature 1..20: note = Synthetic
  source 1..20: mol_type = other DNA
tccacgctagcgctgaagta

SEQ ID NO: 11 - DNA, 14 bp, synthetic construct
  misc_feature 1..14: note = Synthetic
  source 1..14: mol_type = other DNA
ctcagcggaagcta

SEQ ID NO: 12 - DNA, 19 bp, synthetic construct
  misc_feature 1..19: note = Synthetic
  source 1..19: mol_type = other DNA
tgactgtcaaaggccacgg

SEQ ID NO: 13 - DNA, 24 bp, synthetic construct
  misc_feature 1..24: note = Synthetic
  source 1..24: mol_type = other DNA
agatggacaagtctaggttccacc

SEQ ID NO: 14 - DNA, 28 bp, synthetic construct
  misc_feature 1..28: note = Synthetic
  source 1..28: mol_type = other DNA
ccgtttagcgcgtgtttacaacaagctg

SEQ ID NO: 15 - DNA, 24 bp, synthetic construct
  misc_feature 1..24: note = Synthetic
  source 1..24: mol_type = other DNA
catcgtgaaccactacatcgagac

SEQ ID NO: 16 - DNA, 18 bp, synthetic construct
  misc_feature 1..18: note = Synthetic
  source 1..18: mol_type = other DNA
gtcgatccactcctgcgg

SEQ ID NO: 17 - DNA, 22 bp, synthetic construct
  misc_feature 1..22: note = Synthetic
  source 1..22: mol_type = other DNA
accgtgaacttccgcaccgagc

SEQ ID NO: 18 - DNA, 23 bp, synthetic construct
  misc_feature 1..23: note = Synthetic
  source 1..23: mol_type = other DNA
ttggactagaaatctcgtgctga

SEQ ID NO: 19 - DNA, 22 bp, synthetic construct
  misc_feature 1..22: note = Synthetic
  source 1..22: mol_type = other DNA
gctacatagggagccttgtcct

SEQ ID NO: 20 - DNA, 16 bp, synthetic construct
  misc_feature 1..16: note = Synthetic
  source 1..16: mol_type = other DNA
gcgtttgtgtggattg

SEQ ID NO: 21 - DNA, 72 bp, synthetic construct
  misc_feature 1..72: note = Synthetic
  source 1..72: mol_type = other DNA
gcatctaggaccgactagctaactaactagggcgccatgaggagcaatcattgttcaagacatgatgcaaag

SEQ ID NO: 22 - DNA, 57 bp, synthetic construct
  misc_feature 1..57: note = Synthetic
  source 1..57: mol_type = other DNA
gctggccgtgaaggtgaagctcagcggaagctacggctacttcagcgctagcgtgga

SEQ ID NO: 23 - DNA, 113 bp, synthetic construct
  misc_feature 1..113: note = Synthetic
  source 1..113: mol_type = other DNA
tgactgtcaaaggccacggccgtttagcgcgtgtttacaacaagctgtaagagcttactgaaaaaattaacatctcttgctaagctgggggtggaacctagacttgtccatct

SEQ ID NO: 24 - DNA, 76 bp, synthetic construct
  misc_feature 1..76: note = Synthetic
  source 1..76: mol_type = other DNA
catcgtgaaccactacatcgagacctccaccgtgaacttccgcaccgagccgcagaccccgcaggagtggatcgac

SEQ ID NO: 25 - DNA, 79 bp, synthetic construct
  misc_feature 1..79: note = Synthetic
  source 1..79: mol_type = other DNA
ttggactagaaatctcgtgctgattaattgttttacgcgtgcgtttgtgtggattgtaggacaaggctccctatgtagc

SEQ ID NO: 26 - DNA, 10 bp, synthetic construct
  misc_feature 1..10: note = Synthetic
  source 1..10: mol_type = other DNA
cgaggggtac

SEQ ID NO: 27 - DNA, 20 bp, synthetic construct
  misc_feature 1..20: note = Synthetic
  source 1..20: mol_type = other DNA
aggatcgaggggtacctcac

SEQ ID NO: 28 - DNA, 50 bp, synthetic construct
  misc_feature 1..50: note = Synthetic
  source 1..50: mol_type = other DNA
ccatggtgcataatgaggatcgaggggtacctcactgactagctaatcga

SEQ ID NO: 29 - DNA, 10 bp, synthetic construct
  misc_feature 1..10: note = Synthetic
  source 1..10: mol_type = other DNA
gcgccatgag

SEQ ID NO: 30 - DNA, 20 bp, synthetic construct
  misc_feature 1..20: note = Synthetic
  source 1..20: mol_type = other DNA
agggcgcgccatgaggagca

SEQ ID NO: 31 - DNA, 50 bp, synthetic construct
  misc_feature 1..50: note = Synthetic
  source 1..50: mol_type = other DNA
actagctaactaactagggcgcgccatgaggagcaatcattgttcaagac

SEQ ID NO: 32 - DNA, 24867 bp, synthetic construct
  misc_feature 1..24867: note = Synthetic
  source 1..24867: mol_type = other DNA
cgtactgcactgccaccattcaagacaagctatggtcgtcgccgtgaacgtgagccgcgcatcgttgaaggctaggtccacgaggaggtagacaagggcacatgcgctcccaagagcttgttcgtgttcgtgacccgacgaccgcgcgcgtttgtccgtgggctcgtgggacgccagcagcccacgcgaaacggttcccctgccgcgcgcgcgcgcgcccggtgcatggctctcctcagcctccgaatggagtcatcaccgtcgacgcctcgacggcgcggaggtgaccaaataaggtccggctgctcgcagccgtggcacgaaacgaggacacacggactggtaggaggctagggggggcacggacaagaccgctccgttccgttgcctctcccgcgctctgcgagggatgatgcatgggcagcgcgcggacacacggactggcagcggccctgtacccacgtcgcagtgccgggatccgcgcctaccacggcacgatcagcgtcatcatggcacaatcattcacgaacctcccagtcccagcggattgcacgccgaaaaatcgtcctctagaagaaagctttttctcgtcgcacatcggcgcttctgctcggcacggcatgtgaaactgtcagggcacaggggtaggtttactgacaaggtttgatttcttttccggctgctgagcacggtccgcgactgggagcaaattcaatgcccttgctttctcccgtcccggcccattcaaaccgggccgtcgttttagtttctttttatgttttacttggttttagcggcgaggcgatgcaacgaagaccacagcagcaaactactattgtttcagggtcatttctaatacactctccgtcctaaaatattaggtggcgcttggcagagtttccacagcttcgctcagagcgagaatcactcttactgagctaaatgataaaaatcggaactgcttcatgttgagagtggagtgaatctagtctacactatgtattagaaagtgagagaaaaaaaacatgcttcccactacactactcccaacatgctccccactctttaacccctcatgaatcatttcacagcctatttgccaaacaattttcgtcaaatatattcacttctagtagagaatcactcagaatcaccaatcagctctcggaactagagaattgatcaaagacaaactctattaccttcaatgatatgtggttatgattcatgttggcaagctgtgatcctgttgctgctgttcccctggtacttttttttttgtgtgccggttgagtttttatagtttatgttgtgctgcatatgaatgagttttggtagttaccgtggttgccgttcacagcggcgttgcctgctgttcatatgttggcaatccgtgaatttgcatgcttctatatatgagtttggtgttcaattgtacttatatactaccaaaaaataccatagtttcagtgggctgaagaaattaaaacccatgcatgctagtaatgtcatagctaggtgtataccatggttttccaataccaaacttagctaccaaacacatactactgcatctgtatatataatgcactggtgtgcagccgtcagccgtgtgccttctgaagcgtgaaacctggtaaaaaaaaaaaacgggggaggcaagaagcatcagggcaggcaggaggatcctagtacatatcctacttactggcttgcagcaaggctagctgatggctgcgtgcgtgcttacatgatgatgatcatgcgtgtcgttcgtactcgtacgaggatcgaggaaagacaccataactcaccttcaacagacacccttcgttaggagcatgcacggatagatggcgtctagcatatcgataggacatgacaagtggtacgatccccgtcacatgtccatggaggcatctgatatggacacggcgtgtatctatcgcggctggaacagaaccagcgctcgcgcggcggtcggcgggagggacagaccttggctccgtgcgttcaggttgtgcttgtgccgcgcgccacgcacggtctccgccgcctgcagctgaaattttagatttacatcctatccctttattttttttatttgtcacaattcagttcaaaaatgaagaacggaggtagtgcatcctttgtgagactaatgaaaatcacatctggatcctgaaatcggcgtcgtaacctacaaggccacggactggattagatagtggtccatggtgcataatgaggatcgaggggtacctcactgactagctaatcgagctagttaccctatgaggtgacatgaagcgctcacggttactatgacggttagcttcacgactgttggtggcagtagcgtacgacttagctatagttccggacttaccgataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataacctaaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctaggcttaggctactgctagtccgcggtgtatccgtgatcgagttggcgccagacggaatctgttctccatcgctgacatcctcgagtagatcacattcaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctgcgcgccgccggctacaagcacggcggctggcacgacgtcggcttctggcagcgcgacttcgagctgccggccccgccgcgcccggtgcgcccggtgacgcagatctgagtcgacctgcaggcatgccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctaatgcggcccgaataacttcgtatagcatacattatacgaagttatacctggtggcgccgctaggggctgcaggaattcctgcagcccgggggatccactagttctagagcggccgacctcgacagatctaagcttactagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcactagtgattcagatctgatatcgatgggcccactaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggtcacccggtccgggcctagaaggccagcttcaagtttgtacaaaaaagcaggctccggccagaatctcactgactagctaaacagcggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatttcgaattttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatgtcgacgggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgttcgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcggtcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccgccatggcagagccgaacaagggtggagcacctgcgatgaagaacgtcgccaagccgtcgaccaaacgcctgatcccgagctcgatagccgcttcgagccagactagcgccaacgctctgacggagccccttcctgggtctgacgcgatcggccagagctacgacgcattcgggttcttcgccaatccccgcagcatcatgaaggagctgttcgagttcagcccacaggaggagatcgtcgtcgaaggcaacacctggcttctcagcagcgacttcgtctacaccgccatccgcgacacagagacctcgaccgtctcgaggcgcaccaaggacgactacagcaaggagctggccgtgaaggtgaagctcagcggaagctacggctacttcagcgctagcgtggagagcgacttcagccagagcatcagcgacgctaccgacacgacgtacaccagcgtccgcacccacgtcaacaagtggcgcctcagcctcaaggacgacgtcggagctctccggagcaagctccttcctggtgtcaagcaggctctcgctacgatggacgcaacccagctcttcgacacgttcggcacccactacgtcagcgaggtcctcgtcggtggaagagccgactacgtcgccaccaccaagaccagcgccttcagctcgagcacctccatcagcgtcgctgcagaggcgtcgtttcagagcatcgcaggaggcgaagtcagccccgagagcaaggtcctcgccgagatgctgcgcgagaactccagcacacggctctacgcactgggaggctcagcactcccgaacatcacggacccagcgacctacaacgcctggctggagagcatcgacaccatcccggtcttctgcggcttcacccagaactccctcaagtcgatcagcgagctcgcggattcagcccaacgcagagacgcactcgcgaaggcatcccagtcgtacatccccagctacgtcactcgcccagcagtcgtgggcctcgaggtcatcatctccgactccaactccgagagccctccatacggctacacccggatcgactacgacctcaaccgcaatgccggaggcaagtacgtcttcctctgctacaagcagaagaacatctccgtcggaggtgacgcagacgcgatcacggacgtcctcgtcgtctacggcaacgaccggaacccaagcgtgccctcaggctacaccaagatcgacaaggacctgaactccggagctggagggaagtacatctacttctgctactccaaggacaagcgcaagcaggaggagggccttccgatacgcggacttcgcgtcgttggaccacaccctacgtcagtggcaccgtacggcttcagcaagatcgacatcgacctcaacatgggcgcaggtggggacttcatctacctctgcaagtcgcggcacctcgagtgagttaaccccgggtcaacccatcaggaaggatgaagcgcccctcattttgtgccctaggtcgtggattgctggattttaattttacacatttccttgtcgatcctttctgctgtgtgtggttcgagaatgttagtgtgttatcgcaagatctgggtgtttggaagttatctcattattgggcctcataaattcataattcttgccagttagtgacaactgtagcttaggtttacttctgcttgtagtacatcgcctagatcgtgggagtccctcttttcagacgaatgtcatgaaacattggtttttggaaatgattaggaagacatttgctgttttgtcgactgctgttttttacggccaagttcagagttttttttttcatgtacaaagtatcagcagttaaattatgttacctttaccatggttcttcatatttgttttccttccattgctcaatctatgtcatcttttgaaatggtttggaaggcatcctttataggatatatagatatagatttgaagcataattgttaggataagacaccagctagcctatgctgcaacgcacattattcgacccttaataacaacaggtgatttttatattataaaaaagttggaaaagtatacacaagaatttttcaaaagaagggtaaaagggaacagccctcctgctcgacaattggaattggtgtcccgcataatttttttctgcctttgagaattcaggcgtctctggattctagttcaccatttaccaattagaaggaatactatgtatgtataattctacaatctgcattctacacaatccttctattttctgaatatagttgcaagactagggctctcttatagtatttctaattatagccgctttgcaaagtactgtcatatttgattaggggtattggaaagaaggagaaaagggtgacaccctgcttgacaattggaattgataagcagccagggtaccaaggcgcgaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccattctatcactagctagctgctaattattcccgggcacccagctttcttgtacaaagtggccgttacagaatcactgactagctaatctagcggccgctcaagcttcggcatgcaattcgcatacctacagtacaacgtggccaaagtcatcatttaatgagctctcgggcgcgccgtctcactagctagctgctaacgttcccgggcaactttattatacaaagttgatagatcctacaggccagaatggcctctggattcagcggcctagaaggccgaagtacttggtcttcctaatatcggaccgaggaccgattaaactttaattcggtccgtcaatattcaccgaagcgactaattaactagctgtcccacggcctaactagcacttaatcccctagcctaacctaagagcgctaatctaggctagtggtcacttagggctttaaggctagcgtatacgaagttcctattccgaagttcctattcttcaaaaagtataggaacttctgtacacctgagctgattccgatgacttcgtaggttcctagctcaagccgctcgtgtccaagcgtcacttacgattagctaatgattacggcatctaggaccgactagctaactaactagggcgcgccatgaggagcaatcattgttcaagacatgatgcaaagctagaaaactttgattgtggccgtcctaattgtgaagtttaggccggggggaacttcatgaaccctatcgaagcttaattagttcttttttgttgttagccatgtttgtattgtagtttaggtgaacaacatgacgccgcacccgcgatctcagggctcgtccccacacaggagggcacgtcgtcgtcttcgccgccgagcatcagagattcagagcacgtacacgcacatctcaagcaaacggagtagtacgtcctactcctacgtacatacctagccgacgacctttatgtgcacaccaccactgctctgctgcccggcctctccgtcgtccgttcatcaccagctggtctggtccttcaatttccatgcgtcggtccgaagtcaacattttccgtcaattcatggccaaaggctacaactaacattgtttaatgtcgactgttttttttttgttaatgtgacgccgtgttttttttgttattgctagaacactgtttaatgtcaaactgttttaagtccatgggacgcctcagcaatagtagccttttgttaatgtgacgccgcatcaatcaactaattaaagcccttaaccatctgtgtctcattgttaccggcatcacctaaacaacagatcacggctctacgagcaacgtacataacagtaaactaatggcctgatctgctgggctagtagggccacagcaacactagctgaacgtatgcagcggcggcggcggcaccatccaagtaactcaagcgagcacggttgaacaatacttgaacctttttgtcccaatccactttagaaaactaagaaattcatatcggagaaaggtcgaagaatggtttgagaaaagactcggtagcttccccaccaagtctcacaatagaaacattatgaaaatatttacccttcataaccctaatatgacccttcttcgcagtcgacttctcaaagacgacgtggcttggtttctagggtcgttcttcggcatcaccctcaaaatcaaagtcctcgctatcctcagaatcgttgacattccgaccaatattttataagtaatcttcggaatattactcttctccatagcctcctggaacccagcaagcgcaaactcttcactcttgctcccctcgaccatcatagccaaaaccaaataaccaatgaatctaaaacggtcacaattgataataataaccttcggttttaaagcgctagctattctagcaacaaaataggtgaatggtgtgataattgcgcatacgattttcattctatttataataaaagccaggccagggtcgaataaaaaatataaaactaccactgcaaaatgggcctcgcatttcaagtatctcgatgattagcttaaggaaggtgttttttcaaccttcgacataaggccttcgttcatttctcagtttgagttcgttgcaagaaaacaaactaatactgggaggggctagttttggggccttcatcatttgaaggttctcaaaacactaattaaccattgtttcatgatacatctctaaaagtgttacaggtgcttcagtaaaaaccaccttcgaaataaatcgagcaacgacgaaggtacatttctactagcggataaaactggtcttgggccgaggcagcatacaaagaagcttcgcctgtgcgcgcaagtggcagaggtgttagccgaagtcgataactttggcaacaacagaagaggatggagaaaatacattattatcctgatggtatttgtaaacaatgtttgtaaaaccttggggcatgattataattctatataaagacagttcattttcctataaataggtgagcagtaccctgcataaggcatctttttgaggggtgatcacttcgtttaacttagcttacagagaacctttgtaccatcttgtgtgtggagctgaaggtatgcttgtacacctttatcaagagagatagagtaaattgctaaggcatataagatgagttgaatatgaagttacactttg

(19) United States
(12) Patent Application Publication
(10) Pub. No.: US 2026/0049330 A1
(43) Pub. Date: Feb. 19, 2026

(54) MAIZE EVENT DP-915635-4 AND METHODS FOR DETECTION THEREOF

(71) Applicant: PIONEER HI-BRED INTERNATIONAL, INC., JOHNSTON, IA (US)

(72) Inventors: HEATHER MARIE CHRISTENSEN, ANKENY, IA (US); BIN CONG, JOHNSTON, IA (US); VIRGINIA CRANE, DES MOINES, IA (US); MATTHEW CURTIS HARMON, URBANDALE, IA (US); LUCIANO M JAUREGUY, CLIVE, IA (US); JEFFREY KLEVER, GRIMES, IA (US); ALBERT L LU, WEST DES MOINES, IA (US); KRISTEN DENISE RINEHART KREBS, ANKENY, IA (US); MARGIT C ROSS, JOHNSTON, IA (US)

(73) Assignee: PIONEER HI-BRED INTERNATIONAL, INC., JOHNSTON, IA (US)

(21) Appl. No.: 19/250,376

(22) Filed: Jun. 26, 2025

Related U.S. Application Data
(62) Division of application No. 17/332,245, filed on May 27, 2021, now Pat. No. 12,371,707.
(60) Provisional application No. 63/033,994, filed on Jun. 3, 2020.
(60) Provisional application No. 63/116,192, filed on Nov. 20, 2020.

(51) Int. Cl. C12N 15/82; A01H 1/00; A01H 5/10; A01H 6/46; C12Q 1/6895

(52) U.S. Cl. CPC C12N 15/8286; A01H 1/127; A01H 5/10; A01H 6/4684; C12Q 1/6895; C12Q 2600/13

(57) ABSTRACT

Embodiments disclosed herein relate to the field of plant molecular biology, specifically to DNA constructs for conferring insect resistance to a plant. Embodiments disclosed herein relate to insect resistant corn plant containing event DP-915635-4, and to assays for detecting the presence of event DP-915635-4 in samples and compositions thereof.

### REFERENCE TO SEQUENCE LISTING SUBMITTED ELECTRONICALLY

The official copy of the sequence listing is submitted electronically via EFS-Web as an XML formatted sequence listing with a file named “107506-US-DIV-1 Sequence Listing.xml” created on Jun. 24, 2025, and having a size of 188,000 bytes and is filed concurrently with the specification. The XML formatted sequence listing is based on the ASCII formatted sequence listing originally created on Nov. 13, 2020. The sequence listing contained in this XML formatted document is part of the specification and is herein incorporated by reference in its entirety.

### FIELD

Embodiments disclosed herein relate to the field of plant molecular biology, including to DNA constructs for conferring insect resistance to a plant. Embodiments disclosed herein also include insect resistant corn plant containing event DP-915635-4 and assays for detecting the presence of event DP-915635-4 in a sample and compositions thereof.

### BACKGROUND

Corn is an important crop and is a primary food source in many areas of the world. Damage caused by insect pests is a major factor in the loss of the world's corn crops, despite the use of protective measures such as chemical pesticides. In view of this, insect resistance has been genetically engineered into crops such as corn in order to control insect damage and to reduce the need for traditional chemical pesticides. One group of genes which have been utilized for the production of transgenic insect resistant crops is the delta-endotoxin group from Bacillus thuringiensis (Bt). Delta-endotoxins have been successfully expressed in crop plants such as cotton, potatoes, rice, sunflower, as well as corn, and in certain circumstances have proven to provide excellent control over insect pests. (Perlak, F. J et al. (1990) Bio/Technology 8:939-943; Perlak, F. J. et al. (1993) Plant Mol. Biol. 22:313-321; Fujimoto, H. et al. (1993) Bio/Technology 11:1151-1155; Tu et al. (2000) Nature Biotechnology 18:1101-1104; PCT publication WO 01/13731; and Bing, J. W. et al. (2000) Efficacy of CrylF Transgenic Maize, 14th Biennial International Plant Resistance to Insects Workshop, Fort Collins, CO).

The expression of transgenes in plants is known to be influenced by many different factors, including the orientation and composition of the cassettes driving expression of the individual genes of interest, and the location in the plant genome, perhaps due to chromatin structure (e.g., heterochromatin) or the proximity of transcriptional regulatory elements (e.g., enhancers) close to the integration site (Weising et al. (1988) Ann. Rev. Genet. 22:421-477).

It would be advantageous to be able to detect the presence of a particular event in order to determine whether progeny of a sexual cross contain a transgene of interest.

It is possible to detect the presence of a transgene by a nucleic acid detection method by, e.g., a polymerase chain reaction (PCR) or DNA hybridization using nucleic acid probes. These detection methods generally focus on frequently used genetic elements, such as promoters, terminators, marker genes, etc., because for many DNA constructs, the coding region is interchangeable. As a result, such methods may not be useful for discriminating between different events, particularly those produced using the same DNA construct or very similar constructs unless the DNA sequence of the flanking DNA adjacent to the inserted heterologous DNA is known

### SUMMARY

The embodiments relate to the insect resistant corn (Zea mays) plant event DP-915635-4, also referred to as “maize line DP-915635-4,” “maize event DP-915635-4,” and “DP-915635-4 maize,” to the DNA plant expression construct of corn plant event DP-915635-4, and to methods and compositions for the detection of the transgene construct, flanking, and insertion (the target locus) regions in corn plant event DP-915635-4 and progeny thereof.

In one aspect compositions and methods relate to methods for producing and selecting an insect resistant monocot crop plant. Compositions include a DNA construct that when expressed in plant cells and plants confers resistance to insects. In one aspect, a DNA construct, capable of introduction into and replication in a host cell, is provided that when expressed in plant cells and plants confers insect resistance to the plant cells and plants. Maize event DP-915635-4 was produced by Agrobacterium-mediated transformation with plasmid PHP83175. As described herein, these events include the IPD079Ea (polynucleotide SEQ ID NO: 4 and amino acid SEQ ID NO: 5) cassette (Table 1), which confers resistance to certain Coleopteran plant pests. The insect control components have demonstrated efficacy against Coleopteran insect species, particularly western corn rootworm (WCR). In some embodiments, a polynucleotide encoding an IPD079Ea polypeptide comprises a sequence having at least 95%, 96%, 97%, 98%, or 99% sequence identity to SEQ ID NO: 4. In some embodiments, an IPD079EA polypeptide comprises a sequence having 95%, 96%, 97%, 98%, or 99% sequence identity to SEQ ID NO: 5.

According to some embodiments, compositions and methods are provided for identifying a novel corn plant designated DP-915635-4 (ATCC Deposit Number PTA-126746). The methods are based on primers or probes which specifically recognize 5′ and/or 3′ flanking sequence of DP-915635-4. DNA molecules are provided that comprise primer sequences that when utilized in a PCR reaction will produce amplicons unique to the transgenic event DP-915635-4. In one embodiment, the corn plant and seed comprising these molecules is contemplated. Further, kits utilizing these primer sequences for the identification of the DP-915635-4 event are provided.

Some embodiments relate to specific flanking sequences of DP-915635-4 as described herein, which can be used to develop identification methods for DP-915635-4 in biological samples. More particularly, the disclosure relates to 5′ and/or 3′ flanking regions of DP-915635-4, which can be used for the development of specific primers and probes. Further embodiments relate to identification methods for the presence of DP-915635-4 in biological samples based on the use of such specific primers or probes.

According to some embodiments, methods of detecting the presence of DNA corresponding to the corn event DP-915635-4 in a sample are provided. Such methods comprise: (a) contacting the sample comprising DNA with a DNA primer set, that when used in a nucleic acid amplification reaction with genomic DNA extracted from corn comprising event DP-915635-4 produces an amplicon that is diagnostic for corn event DP-915635-4, respectively; (b) performing a nucleic acid amplification reaction, thereby producing the amplicon; and (c) detecting the amplicon. In some aspects, the primer set comprises SEQ ID NOs: 6 and 7, and optionally a probe comprising SEQ ID NO: 8.

According to some embodiments, methods of detecting the presence of a DNA molecule corresponding to the DP-915635-4 event in a sample comprise: (a) contacting the sample comprising DNA extracted from a corn plant with a DNA probe molecule that hybridizes under stringent hybridization conditions with DNA extracted from corn event DP-915635-4 and does not hybridize under the stringent hybridization conditions with a control corn plant DNA; (b) subjecting the sample and probe to stringent hybridization conditions; and (c) detecting hybridization of the probe to the DNA extracted from corn event DP-915635-4. More specifically, a method for detecting the presence of a DNA molecule corresponding to the DP-915635-4 event in a sample consist of (a) contacting the sample comprising DNA extracted from a corn plant with a DNA probe molecule that comprises sequences that are unique to the event, e.g. junction sequences, wherein said

DNA probe molecule hybridizes under stringent hybridization conditions with DNA extracted from corn event DP-915635-4 and does not hybridize under the stringent hybridization conditions with a control corn plant DNA; (b) subjecting the sample and probe to stringent hybridization conditions; and (c) detecting hybridization of the probe to the DNA.

In addition, a kit and methods for identifying event DP-915635-4 in a biological sample which detects a DP-915635-4 specific region are provided.

DNA molecules are provided that comprise at least one junction sequence of DP-915635-4; wherein a junction sequence spans the junction located between heterologous DNA inserted into the genome and the DNA from the maize cell flanking the insertion site and may be diagnostic for the DP-915635-4 event.

According to some embodiments, methods of producing an insect resistant corn plant comprise the steps of: (a) sexually crossing a first parental corn line comprising the expression cassettes disclosed herein, which confer resistance to insects, and a second parental corn line that lacks such expression cassettes, thereby producing a plurality of progeny plants; and (b) selecting a progeny plant that is insect resistant. Such methods may optionally comprise the further step of back-crossing the progeny plant to the second parental corn line to produce a true-breeding corn plant that is insect resistant.

Some embodiments provide a method of producing a corn plant that is resistant to insects comprising transforming a corn cell with the DNA construct PHP74643, growing the transformed corn cell into a corn plant, selecting the corn plant that shows resistance to insects, and further growing the corn plant into a fertile corn plant. The fertile corn plant can be self-pollinated or crossed with compatible corn varieties to produce insect resistant progeny.

Some embodiments further relate to a DNA detection kit for identifying maize event DP-915635-4 in biological samples. The kit comprises a first primer which specifically recognizes the 5′ or 3′ flanking region of DP-915635-4, and a second primer which specifically recognizes a sequence within the non-native target locus DNA of DP-915635-4, respectively, or within the flanking DNA, for use in a PCR identification protocol. A further embodiment relates to a kit for identifying event DP-915635-4 in biological samples, which kit comprises a specific probe having a sequence which corresponds or is complementary to, a sequence having between about 80% and 100% sequence identity with a specific region of event DP-915635-4. The sequence of the probe corresponds to a specific region comprising part of the 5′ or 3′ flanking region of event DP-915635-4. In some embodiments, the first or second primer comprises any one of SEQ ID NOs: 6-7, 9-10, 12-13, 15-16, or 18-19.

The methods and kits encompassed by the embodiments disclosed herein can be used for different purposes such as, but not limited to the following: to identify event DP-915635-4 in plants, plant material or in products such as, but not limited to, food or feed products (fresh or processed) comprising, or derived from plant material; additionally or alternatively, the methods and kits can be used to identify transgenic plant material for purposes of segregation between transgenic and non-transgenic material; additionally or alternatively, the methods and kits can be used to determine the quality of plant material comprising maize event DP-915635-4. The kits may also contain the reagents and materials necessary for the performance of the detection method.

A further embodiment relates to the DP-915635-4 maize plant or its parts, including, but not limited to, pollen, ovules, vegetative cells, the nuclei of pollen cells, and the nuclei of egg cells of the corn plant DP-915635-4 and the progeny derived thereof. In another embodiment, the DNA primer molecules targeting the maize plant and seed of DP-915635-4 provide a specific amplicon product

### DESCRIPTION OF THE DRAWINGS

FIG. 1. shows a schematic diagram of plasmid PHP83175 with genetic elements indicated. Plasmid size is 74,997 bp (SEQ ID NO: 1).

FIG. 2. shows a schematic diagram of the T-DNA indicating six gene cassettes. The T-DNA was used to transform a pre-characterized maize line containing FRT1 and FRT6 sites. The region containing the pmi gene, the mo-pat gene, and the ipd079Ea gene between the FRT1 and FRT6 sites in the T-DNA was integrated into the maize line in a site-specific manner. The zm-wus2 and zm-odp2 developmental genes were present to increase transformation efficiency. The zm-wus2, zm-odp2, and mo-Flp genes were not incorporated in the final product.

FIG. 3. shows a schematic map of the insertion in DP915635 maize based on the SbS analysis described. The flanking maize genome (including the zm-SEQ158 and zm-SEQ159 regions) is represented by the horizontal black bars. A single copy of the intended insertion, derived from PHP83175 and PHP73878, is integrated into the maize genome (SEQ ID NO: 2 is the insert T-DNA sequence). Within the insertion, the landing pad sequences from PHP73878 and the trait genes derived from PHP83175 are highlighted. SEQ ID NO: 3 is the complete insert sequence and flanking regions. The FRT1 and FRT6 sites that are the targets of recombination during the SSI process are highlighted.

FIG. 4. shows a schematic Diagram of the Transformation and Development of DP-915635-4.

FIG. 5 is a table showing hybrid performance of event DP-915635-4 compared to a base entry for non-yield agronomic traits.

FIG. 6 is a table showing inbred performance of event DP-915635-4 compared to a base entry for all agronomic traits.

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

Compositions of this disclosure include seed deposited as ATCC Patent Deposit No. PTA-126746 and plants, plant cells, and seed derived therefrom. Applicant(s) deposited at least 2500 seeds of maize event DP-915635-4 (Patent Deposit No. PTA-126746) with the American Type Culture Collection (ATCC), Manassas, VA 20110-2209 USA, on Mar. 27, 2020. These deposits will be maintained under the terms of the Budapest Treaty on the International Recognition of the Deposit of Microorganisms for the Purposes of Patent Procedure. The seeds deposited with the ATCC on Mar. 27, 2020 were taken from the deposit maintained by Pioneer Hi-Bred International, Inc., 7250 NW 62^{nd} Avenue, Johnston, Iowa 50131-1000. Access to this deposit will be available during the pendency of the application to the Commissioner of Patents and Trademarks and persons determined by the Commissioner to be entitled thereto upon request. Upon allowance of any claims in the application, the Applicant(s) will make available to the public, pursuant to 37 C.F.R. § 1.808, sample(s) of the deposit of at least 625 seeds of hybrid maize with the American Type Culture Collection (ATCC), 10801 University Boulevard, Manassas, VA 20110-2209. This deposit of seed of maize event DP-915635-4 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant(s) have satisfied all the requirements of 37 C.F.R. §§ 1.801-1.809, including providing an indication of the viability of the sample upon deposit. Applicant(s) have no authority to waive any restrictions imposed by law on the transfer of biological material or its transportation in commerce. Applicant(s) do not waive any infringement of their rights granted under this patent or rights applicable to event DP-915635-4 under the Plant Variety Protection Act (7 USC 2321 et seq.). Unauthorized seed multiplication is prohibited. The seed may be regulated.

A first cassette contains the insecticidal protein gene, IPD079Ea, from Ophioglossum pendulum (international patent application publication number WO 2017023486). The expressed IPD079Ea protein in plants is effective against certain coleopteran pests. The IPDO79Ea protein is 479 amino acids in length and has a molecular weight of approximately 52 kDa. Expression of the IPDO79Ea gene is controlled by three copies of the enhancer region, showing root-specific activity, from the sorghum (Sorghum bicolor) root cortical RCc3 (sb-RCc3) gene (international patent application publication number WO 2012112411) followed by the promoter region upstream of a Zea mays PCO118362 mRNA sequence (zm PCOa) identified as having root-specific activity (international patent application publication number WO 2017222821) and the intron region from the Zea mays ortholog of a rice (Oryza sativa) hypothetical protein (zm-HPLV9) gene, a predicted Zea mays calmodulin 5 gene (Phytozome gene ID Zm00008a029682; international patent application publication number WO 2016109157). The terminator for the IPDO79Ea gene is the terminator region from the sorghum (Sorghum bicolor) subtilisin-chymotrypsin inhibitor 1B (sb-SCI-1B) gene (international patent application publication number WO 2018102131). Three additional terminators are present to prevent transcriptional interference: the terminator region from the maize W64 line 27-kDa gamma zein (Z27G) gene (Das et al., 1991; Liu et al., 2016), the terminator region from the Arabidopsis thaliana ubiquitin 14 (UBQ14) gene (Callis et al., 1995), and the terminator region from the maize In2-1 gene (Hershey and Stoner, 1991).

A second gene cassette (mo-pat gene cassette) contains the phosphinothricin acetyl transferase gene (mo-pat) from Streptomyces viridochromogenes (Wohlleben et al., 1988). The mo-pat gene expresses the phosphinothricin acetyl transferase (PAT) enzyme that confers tolerance to phosphinothricin. The PAT protein is 183 amino acids in length and has a molecular weight of approximately 21 kDa. Expression of the mo-pat gene is controlled by the promoter and intron region of the Oryza sativa (rice) actin (os-actin) gene (GenBank accession CP018159), in conjunction with a third copy of the CaMV35S terminator. Two additional terminators are present to prevent transcriptional interference: the terminator regions from the Sorghum bicolor (sorghum) ubiquitin (sb-ubi) gene (Phytozome gene ID Sobic.004G049900.1) and γ-kafarin (sb-gkaf) gene (de Freitas et al., 1994), respectively.

A third gene cassette (pmi gene cassette) contains the phosphomannose isomerase (pmi) gene from Escherichia coli (Negrotto et al., 2000). Expression of the PMI protein in plants serves as a selectable marker which allows plant tissue growth with mannose as the carbon source. The PMI protein is 391 amino acids in length and has a molecular weight of approximately 43 kDa. As present in the T-DNA region of PHP74643, the pmi gene lacks a promoter, but its location next to the flippase recombination target site, FRT1, allows post-recombination expression by an appropriately-placed promoter. The terminator for the pmi gene is a fourth copy of the pinII terminator. An additional Z19 terminator present is intended to prevent transcriptional interference between cassettes.

As used herein, the term “corn” means Zea mays or maize and includes all plant varieties that can be bred with corn, including wild maize species.

As used herein, the terms “insect resistant” and “impacting insect pests” refers to effecting changes in insect feeding, growth, and/or behavior at any stage of development, including but not limited to: killing the insect; retarding growth; reducing reproductive capability; inhibiting feeding; and the like.

As used herein, the terms “pesticidal activity” and “insecticidal activity” are used synonymously to refer to activity of an organism or a substance (such as, for example, a protein) that can be measured by numerous parameters including, but not limited to, pest mortality, pest weight loss, pest attraction, pest repellency, and other behavioral and physical changes of a pest after feeding on and/or exposure to the organism or substance for an appropriate length of time. For example, “pesticidal proteins” are proteins that display pesticidal activity by themselves or in combination with other proteins.

As used herein, “insert DNA” refers to the heterologous DNA within the expression cassettes used to transform the plant material while “flanking DNA” can exist of either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 10 bp (in some narrower embodiments, at least 20 bp, at least 50 bp, and up to at least 5000 bp), which is located either immediately upstream of and contiguous with and/or immediately downstream of and contiguous with the original non-native insert DNA molecule. Transformation procedures of the foreign DNA may result in transformants containing different flanking regions characteristic and unique for each transformant. When recombinant DNA is introduced into a plant through traditional crossing, its flanking regions will generally not be changed. It may be possible for single nucleotide changes to occur in the flanking regions through generations of plant breeding and traditional crossing.

Transformants will also contain unique junctions between a piece of heterologous insert DNA and genomic DNA, or two (2) pieces of genomic DNA, or two (2) pieces of heterologous DNA. A “junction” is a point where two (2) specific DNA fragments join. For example, a junction exists where insert DNA joins flanking DNA. A junction point also exists in a transformed organism where two (2) DNA fragments join together in a manner that is modified from that found in the native organism. “Junction DNA” refers to DNA that comprises a junction point. Junction sequences set forth in this disclosure include a junction point located between the maize genomic DNA and the 5′ end of the insert, which range from at least-5 to +5 nucleotides of the junction point (SEQ ID NO: 26), from at least-10 to +10 nucleotides of the junction point (SEQ ID NO: 27), and from at least-25 to +25 nucleotides of the junction point (SEQ ID NO: 28); and a junction point located between the 3′ end of the insert and maize genomic DNA, which range from at least −5 to +5 nucleotides of the junction point (SEQ ID NO: 29), from at least-10 to +10 nucleotides of the junction point (SEQ ID NO: 30), and from at least-25 to +25 nucleotides of the junction point (SEQ ID NO: 31). Junction sequences set forth in this disclosure also include a junction point located between the target locus and the 5′ end of the insert. In some embodiments, SEQ ID NOs: 8 or 21 for DP-915635-4 represent the junction point located between the target locus and the 5′ end of the insert. The complete insert with flanking regions is represented in SEQ ID NO: 3. In some embodiments, the insert and flanking regions comprise a polynucleotide having at least 95%, 96%, 97%, 98%, or 99% sequence identity compared to SEQ ID NO: 3.

As used herein, “heterologous” in reference to a nucleic acid sequence is a nucleic acid sequence that originates from a different non-sexually compatible species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

“Promoter” refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, or even comprise synthetic nucleotide segments. It is understood by those skilled in the art that different regulatory elements may direct the expression of a gene in different tissues or cell types, or at different stages of development, or in response to different environmental conditions. Promoters that cause a nucleic acid fragment to be expressed in most cell types at most times are commonly referred to as “constitutive promoters”. It is further recognized that since in most cases the exact boundaries of regulatory sequences have not been completely defined, nucleic acid fragments of different lengths may have identical or similar promoter activity.

The “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, processing of the primary transcript to mRNA, mRNA stability and/or translation efficiency.

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

A DNA construct is an assembly of DNA molecules linked together that provide one or more expression cassettes. The DNA construct may be a plasmid that is enabled for self-replication in a bacterial cell and contains various endonuclease enzyme restriction sites that are useful for introducing DNA molecules that provide functional genetic elements, i.e., promoters, introns, leaders, coding sequences, 3′ termination regions, among others; or a DNA construct may be a linear assembly of DNA molecules, such as an expression cassette. The expression cassette contained within a DNA construct comprises the necessary genetic elements to provide transcription of a messenger RNA. The expression cassette can be designed to express in prokaryotic cells or eukaryotic cells. Expression cassettes of the embodiments are designed to express in plant cells.

The DNA molecules disclosed herein are provided in expression cassettes for expression in an organism of interest. The cassette includes 5′ and 3′ regulatory sequences operably linked to a coding sequence. “Operably linked” means that the nucleic acid sequences being linked are contiguous and, where necessary to join two protein coding regions, contiguous and in the same reading frame. Operably linked is intended to indicate a functional linkage between a promoter and a second sequence, wherein the promoter sequence initiates and mediates transcription of the DNA sequence corresponding to the second sequence. The cassette may additionally contain at least one additional gene to be co-transformed into the organism. Alternatively, the additional gene(s) can be provided on multiple expression cassettes or multiple DNA constructs.

The expression cassette may include in the 5′ to 3′ direction of transcription: a transcriptional and translational initiation region, a coding region, and a transcriptional and translational termination region functional in the organism serving as a host. The transcriptional initiation region (e.g., the promoter) may be native or analogous, or foreign or heterologous to the host organism. Additionally, the promoter may be the natural sequence or alternatively a synthetic sequence. The expression cassettes may additionally contain 5′ leader sequences in the expression cassette construct. Such leader sequences can act to enhance translation.

It is to be understood that as used herein the term “transgenic” generally includes any cell, cell line, callus, tissue, plant part, or plant, the genotype of which has been altered by the presence of a heterologous nucleic acid including those initially so altered as well as those created by sexual crosses or asexual propagation from the initial transgenic and retains such heterologous nucleic acids.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety, wherein the progeny includes the heterologous DNA. After back-crossing to a recurrent parent, the inserted DNA and the linked flanking genomic DNA from the transformed parent is present in the progeny of the cross at the same chromosomal location. A progeny plant may contain sequence changes to the insert arising as a result of conventional breeding techniques. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

An insect resistant DP-915635-4 corn plant may be bred by first sexually crossing a first parental corn plant having the transgenic DP-915635-4 event plant and progeny thereof derived from transformation with the expression cassettes of the embodiments that confers insect resistance, and a second parental corn plant that lacks such expression cassettes, thereby producing a plurality of first progeny plants; and then selecting a first progeny plant that is resistant to insects; and selfing the first progeny plant, thereby producing a plurality of second progeny plants; and then selecting from the second progeny plants an insect resistant plant. These steps can further include the back-crossing of the first insect resistant progeny plant or the second insect resistant progeny plant to the second parental corn plant or a third parental corn plant, thereby producing a corn plant that is resistant to insects. The term “selfing” refers to self-pollination, including the union of gametes and/or nuclei from the same organism.

As used herein, the term “plant” includes reference to whole plants, parts of plants, plant organs (e.g., leaves, stems, roots, etc.), seeds, plant cells, and progeny of same. In some embodiments, parts of transgenic plants comprise, for example, plant cells, protoplasts, tissues, callus, embryos as well as flowers, stems, fruits, leaves, and roots originating in transgenic plants or their progeny previously transformed with a DNA molecule disclosed herein, and therefore consisting at least in part of transgenic cells.

As used herein, the term “plant cell” includes, without limitation, seeds, suspension cultures, embryos, meristematic regions, callus tissue, leaves, roots, shoots, gametophytes, sporophytes, pollen, and microspores. The class of plants that may be used is generally as broad as the class of higher plants amenable to transformation techniques, including both monocotyledonous and dicotyledonous plants.

“Transformation” refers to the transfer of a nucleic acid fragment into the genome of a host organism, resulting in genetically stable inheritance. Host plants containing the transformed nucleic acid fragments are referred to as “transgenic” plants.

As used herein, the term “progeny,” in the context of event DP-915635-4, denotes an offspring of any generation of a parent plant which comprises corn event DP-915635-4.

Isolated polynucleotides disclosed herein may be incorporated into recombinant constructs, typically DNA constructs, which are capable of introduction into and replication in a host cell. Such a construct may be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., (1985; Supp. 1987) Cloning Vectors: A Laboratory Manual, Weissbach and Weissbach (1989) Methods for Plant Molecular Biology, (Academic Press, New York); and Flevin et al., (1990) Plant Molecular Biology Manual, (Kluwer Academic Publishers). Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally-or developmentally-regulated, or cell- or tissue-specific expression), a transcription initiation start site, a ribosome binding site, an RNA processing signal, a transcription termination site, and/or a polyadenylation signal.

During the process of introducing an insert into the genome of plant cells, it is not uncommon for some deletions or other alterations of the insert and/or genomic flanking sequences to occur. Thus, the relevant segment of the plasmid sequence provided herein might comprise some minor variations. The same is possible for the flanking sequences provided herein. Thus, a plant comprising a polynucleotide having some range of identity with the subject flanking and/or insert sequences is within the scope of the subject disclosure. Identity to the sequence of the present disclosure may be a polynucleotide sequence having at least 65% sequence identity, at least 70% sequence identity, at least 75% sequence identity at least 80% identity, or at least 85% 86%, 87%, 88%, 89%, 90%, 91%, 92%, 93%, 94%, 95%, 96%, 97%, 98%, 99% sequence identity with a sequence exemplified or described herein. Hybridization and hybridization conditions as provided herein can also be used to define such plants and polynucleotide sequences of the subject disclosure. A sequence comprising the flanking sequences plus the full insert sequence can be confirmed with reference to the deposited seed.

In some embodiments, two different transgenic plants can also be crossed to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation.

A “probe” is an isolated nucleic acid to which is attached a conventional, synthetic detectable label or reporter molecule, e.g., a radioactive isotope, ligand, chemiluminescent agent, or enzyme. Such a probe is complementary to a strand of a target nucleic acid, for example, to a strand of isolated DNA from corn event DP-915635-4 whether from a corn plant or from a sample that includes DNA from the event. Probes may include not only deoxyribonucleic or ribonucleic acids but also polyamides and other modified nucleotides that bind specifically to a target DNA sequence and can be used to detect the presence of that target DNA sequence.

“Primers” are isolated nucleic acids that anneal to a complementary target DNA strand by nucleic acid hybridization to form a hybrid between the primer and the target DNA strand, then extended along the target DNA strand by a polymerase, e.g., a DNA polymerase. Primer pairs refer to their use for amplification of a target nucleic acid sequence, e.g., by PCR or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence specifically in the hybridization conditions or reaction conditions determined by the operator. This length may be of any length that is of sufficient length to be useful in a detection method of choice. Generally, 11 nucleotides or more in length, 18 nucleotides or more, and 22 nucleotides or more, are used. Such probes and primers hybridize specifically to a target sequence under high stringency hybridization conditions. Probes and primers according to embodiments may have complete DNA sequence similarity of contiguous nucleotides with the target sequence, although probes differing from the target DNA sequence and that retain the ability to hybridize to target DNA sequences may be designed by conventional methods. Probes can be used as primers, but are generally designed to bind to the target DNA or RNA and are not used in an amplification process.

Specific primers may be used to amplify an integration fragment to produce an amplicon that can be used as a “specific probe” for identifying event DP-915635-4 in biological samples. When the probe is hybridized with the nucleic acids of a biological sample under conditions which allow for the binding of the probe to the sample, this binding can be detected and thus allow for an indication of the presence of event DP-915635-4 in the biological sample. In an embodiment of the disclosure, the specific probe is a sequence which, under appropriate conditions, hybridizes specifically to a region within the 5′ or 3′ flanking region of the event and also comprises a part of the foreign DNA contiguous therewith. The specific probe may comprise a sequence of at least 80%, from 80 and 85%, from 85 and 90%, from 90 and 95%, and from 95 and 100% identical (or complementary) to a specific region of the event.

Methods for preparing and using probes and primers are described, for example, in Sambrook et al., Molecular Cloning: A Laboratory Manual, 2^{nd} ed., vol. 1-3, Cold Spring Harbor Laboratory Press, Cold Spring Harbor, N.Y. 1989 (hereinafter, “Sambrook et al., 1989”); Ausubel et al. eds., Current Protocols in Molecular Biology, Greene Publishing and Wiley-Interscience, New York, 1995 (with periodic updates) (hereinafter, “Ausubel et al., 1995”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 6 (Informax Inc., Bethesda MD); PrimerSelect (DNASTAR Inc., Madison, WI); and Primer (Version 0.50, 1991, Whitehead Institute for Biomedical Research, Cambridge, Mass.). Additionally, the sequence can be visually scanned and primers manually identified using guidelines known to one of skill in the art.

A “kit” as used herein refers to a set of reagents, and optionally instructions, for the purpose of performing method embodiments of the disclosure, more particularly, the identification of event DP-915635-4 in biological samples. A kit may be used, and its components can be specifically adjusted, for purposes of quality control (e.g. purity of seed lots), detection of event DP-915635-4 in plant material, or material comprising or derived from plant material, such as but not limited to food or feed products. “Plant material” as used herein refers to material which is obtained or derived from a plant.

Primers and probes based on the flanking DNA and insert sequences disclosed herein can be used to confirm (and, if necessary, to correct) the disclosed sequences by conventional methods, e.g., by re-cloning and sequencing such sequences. The nucleic acid probes and primers hybridize under stringent conditions to a target DNA sequence. Any conventional nucleic acid hybridization or amplification method may be used to identify the presence of DNA from a transgenic event in a sample.

A nucleic acid molecule is said to be the “complement” of another nucleic acid molecule if they exhibit complete complementarity or minimal complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, a Practical Approach, IRL Press, Washington, D.C. (1985), departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

In hybridization reactions, specificity is typically the function of post-hybridization washes, the critical factors being the ionic strength and temperature of the final wash solution. The thermal melting point (Tₘ) is the temperature (under defined ionic strength and pH) at which 50% of a complementary target sequence hybridizes to a perfectly matched probe. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH.

However, in some embodiments, other stringency conditions can be applied, including severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ.

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), a user may choose to increase the SSC concentration so that a higher temperature can be used. An extensive guide to the hybridization of nucleic acids is found in Tijssen (1993) Laboratory Techniques in Biochemistry and Molecular Biology Hybridization with Nucleic Acid Probes, Part I, Chapter 2 (Elsevier, New York); and Ausubel et al., eds. (1995) and Sambrook et al. (1989).

In some embodiments, a complementary sequence has the same length as the nucleic acid molecule to which it hybridizes. In some embodiments, the complementary sequence is 1, 2, 3, 4, 5, 6, 7, 8, 9, or 10 nucleotides longer or shorter than the nucleic acid molecule to which it hybridizes. In some embodiments, the complementary sequence is 1%, 2%, 3%, 4%, or 5% longer or shorter than the nucleic acid molecule to which it hybridizes. In some embodiments, a complementary sequence is complementary on a nucleotide-for-nucleotide basis, meaning that there are no mismatched nucleotides (each A pairs with a T and each G pairs with a C). In some embodiments, a complementary sequence comprises 1, 2, 3, 4, 5, 6, 7, 8, 9, 10 or less mismatches. In some embodiments, the complementary sequence comprises 1%, 2%, 3%, 4%, 5%, 6%, 7%, 8%, 9%, or 10% or less mismatches.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Regarding the amplification of a target nucleic acid sequence (e.g., by PCR) using a particular amplification primer pair, stringent conditions permit the primer pair to hybridize only to the target nucleic-acid sequence to which a primer having the corresponding wild-type sequence (or its complement) would bind and optionally to produce a unique amplification product, the amplicon, in a DNA thermal amplification reaction.

As used herein, “amplified DNA” or “amplicon” refers to the product of nucleic acid amplification of a target nucleic acid sequence that is part of a nucleic acid template. For example, to determine whether a corn plant resulting from a sexual cross contains transgenic event genomic DNA from the corn plant disclosed herein, DNA extracted from a tissue sample of a corn plant may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol. Alternatively, primer pairs can be derived from flanking sequence on both sides of the inserted DNA so as to produce an amplicon that includes the entire insert nucleotide sequence of the PHP74643 expression construct as well as a portion of the sequence flanking the transgenic insert. A member of a primer pair derived from the flanking sequence may be located a distance from the inserted DNA sequence, this distance can range from one nucleotide base pair up to the limits of the amplification reaction. The use of the term “amplicon” specifically excludes primer dimers that may be formed in the DNA thermal amplification reaction.

Nucleic acid amplification can be accomplished by any of the various nucleic acid amplification methods known in the art, including PCR. A variety of amplification methods are known in the art and are described, inter alia, in U.S. Pat. Nos. 4,683,195 and 4,683,202 and in Innis et al., (1990) supra. PCR amplification methods have been developed to amplify up to 22 Kb of genomic DNA and up to 42 Kb of bacteriophage DNA (Cheng et al., Proc. Natl. Acad. Sci. USA 91:5695-5699, 1994). These methods as well as other methods known in the art of DNA amplification may be used in the practice of the embodiments of the present disclosure. It is understood that a number of parameters in a specific PCR protocol may need to be adjusted to specific laboratory conditions and may be slightly modified and yet allow for the collection of similar results. These adjustments will be apparent to a person skilled in the art.

The amplicon produced by these methods may be detected by a plurality of techniques, including, but not limited to, Genetic Bit Analysis (Nikiforov, et al. Nucleic Acid Res. 22:4167-4175, 1994) where a DNA oligonucleotide is designed which overlaps both the adjacent flanking DNA sequence and the inserted DNA sequence. The oligonucleotide is immobilized in wells of a microwell plate. Following PCR of the region of interest (for example, using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the pyrosequencing technique as described by Winge (2000) Innov. Pharma. Tech. 00:18-24. In this method an oligonucleotide is designed that overlaps the adjacent DNA and insert DNA junction. The oligonucleotide is hybridized to a single-stranded PCR product from the region of interest (for example, one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence polarization as described by Chen et al., (1999) Genome Res. 9:492-498 is also a method that can be used to detect an amplicon. Using this method an oligonucleotide is designed which overlaps the flanking and inserted DNA junction. The oligonucleotide is hybridized to a single-stranded PCR product from the region of interest (for example, one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

Quantitative PCR (qPCR) is described as a method of detecting and quantifying the presence of a DNA sequence and is fully understood in the instructions provided by commercially available manufacturers. Briefly, in one such qPCR method, a FRET oligonucleotide probe is designed which overlaps the flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular beacons have been described for use in sequence detection as described in Tyangi et al. (1996) Nature Biotech. 14:303-308. Briefly, a FRET oligonucleotide probe is designed that overlaps the flanking and insert DNA junction. The unique structure of the FRET probe results in it containing secondary structure that keeps the fluorescent and quenching moieties in close proximity. The FRET probe and PCR primers (for example, one primer in the insert DNA sequence and one in the flanking sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

A hybridization reaction using a probe specific to a sequence found within the amplicon is yet another method used to detect the amplicon produced by a PCR reaction.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Of interest are larvae and adults of the order Coleoptera including weevils from the families Anthribida, Bruchidae, and Curculionidae including, but not limited to: Anthonomus grandis Boheman (boll weevil); Cylindrocopturus adspersus LeConte (sunflower stem weevil); Diaprepes abbreviatus Linnacus (Diaprepes root weevil); Hypera punctata Fabricius (clover leaf weevil); Lissorhoptrus oryzophilus Kuschel (rice water wecvil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus

Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabacidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp.

including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

In some embodiments the DP-915635-4 maize event may further comprise a stack of additional traits. Plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid).

In some embodiments the DP-915635-4 maize event disclosed herein, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the embodiments can be used to provide a complete agronomic package of improved crop quality with the ability to flexibly and cost effectively control any number of agronomic pests.

In a further embodiment, the DP-915635-4 maize event may be stacked with one or more additional Bt insecticidal toxins, including, but not limited to, a Cry3B toxin disclosed in U.S. Pat. Nos. 8,101,826, 6,551,962, 6,586,365, 6,593,273, and PCT Publication WO 2000/011185; a mCry3B toxin disclosed in U.S. Pat. Nos. 8,269,069, and 8,513,492; a mCry3A toxin disclosed in U.S. Pat. Nos. 8,269,069, 7,276,583 and 8,759,620; or a Cry34/35 toxin disclosed in U.S. Pat. Nos. 7,309,785, 7,524,810, 7,985,893, 7,939,651 and 6,548,291. In a further embodiment, the DP-915635-4 maize event may be stacked with one or more additional transgenic events containing these Bt insecticidal toxins and other Coleopteran active Bt insecticidal traits for example, event MON863 disclosed in U.S. Pat. No. 7,705,216; event MIR604 disclosed in U.S. Pat. No. 8,884,102; event 5307 disclosed in U.S. Pat. No. 9,133,474; event DAS-59122 disclosed in U.S. Pat. No. 7,875,429; event DP-4114 disclosed in U.S. Pat. No. 8,575,434; event MON 87411 disclosed in U.S. Pat. No. 9,441,240; event DP-23211 disclosed in International Patent Application Publication Number WO 2019/209700; and event MON88017 disclosed in U.S. Pat. No. 8,686,230 all of which are incorporated herein by reference. In some embodiments, the DP-915635-4 maize event may be stacked with MON-87429-9 (MON87429 Event); MON87403; MON95379; MON87427; MON87419; MON-00603-6 (NK603); MON-87460-4; LY038; DAS-06275-8; BT176; BT11; MIR162; GA21; MZDT09Y; SYN-05307-1; and DAS-40278-9.

In some embodiments, the disclosed compositions can be introduced into the genome of a plant using genome editing technologies, or previously introduced polynucleotides in the genome of a plant may be edited using genome editing technologies. For example, the disclosed polynucleotides can be introduced into a desired location in the genome of a plant through the use of double-stranded break technologies such as TALENs, meganucleases, zinc finger nucleases, CRISPR-Cas, and the like. For example, the disclosed polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some embodiments, where the disclosed polynucleotide has previously been introduced into a genome, genome editing technologies may be used to alter or modify the introduced polynucleotide sequence. Site specific modifications that can be introduced into the disclosed compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional insecticidally-active proteins in close proximity to the disclosed compositions disclosed herein within the genome of a plant, in order to generate molecular stacks of insecticidally-active proteins.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In some embodiments, a corn plant comprising a DP-915635-4 event may be treated with a seed treatment. In some embodiments, the seed treatment may be a fungicide, an insecticide, or a herbicide.

The following examples are offered by way of illustration and not by way of limitation.

### Examples

### Example 1. Cassette Design for Transgenic Plants Containing Constructs Encoding IPD079Ea

Cassette designs for IPDO79Ea expression used in the molecular stacks to generate commercial track events was chosen based upon efficacy and expression in gene testing transformation experiments. A large number of different regulatory (promoters, introns) and other elements (terminators) were evaluated in gene testing experiments. The large number of different regulatory elements were used to evaluate expression patterns for yield and trait efficacy.

The genetic elements contained in the IPDO79Ea gene cassette of T-DNA Region of the selected event construct, Plasmid PHP83175, are described in Table 1.

TABLE 1
Description of Genetic Elements in the T-DNA Region of Plasmid PHP83175

| | Location on | | | |
|---|---|---|---|---|
| | T-DNA (Base | | Size | |
| | Pair Position) | Genetic Element | (bp) | Description |
| | 20,120-20,157 | Intervening Sequence | 38 | DNA sequence used for cloning |
| ipd079Ea | 20,158-21,738 | sb-RCc3 Enhancer | 1581 | Enhancer region, showing root-specific |
| gene | | | | activity, from the Sorghum bicolor (sorghum) |
| cassette | | | | root cortical RCc3 (sb-RCc3) gene |
| | 21,739-21,744 | Intervening Sequence | 6 | DNA sequence used for cloning |
| | 21,745-23,325 | sb-RCc3 Enhancer | 1581 | Enhancer region, showing root-specific |
| | | | | activity, from the Sorghum bicolor (sorghum) |
| | | | | root corticol RCc3 (sb-RCc3) gene (WO Patent |
| | | | | 2012112411) |
| | 23,326-23,338 | Intervening Sequence | 13 | DNA sequence used for cloning |
| | 23,339-24,922 | sb-RCc3 Enhancer | 1584 | Enhancer region, showing root-specific |
| | | | | activity, from the Sorghum bicolor (sorghum) |
| | | | | root cortical RCc3 (sb-RCc3) gene (WO Patent |
| | | | | 2012112411) |
| | 24,923-25,833 | zm-PCOa Promoter | 911 | Promoter region upstream of a Zea mays |
| | | | | PCO118362 mRNA sequence identified as |
| | | | | having root-specific activity (WO Patent |
| | | | | 2017222821) |
| | 25,834-25,851 | Intervening Sequence | 18 | DNA sequence used for cloning |
| | 25,852-26,707 | zm-HPLV9 Intron | 856 | Intron region from the Zea mays ortholog of an |
| | | | | Oryza sativa (rice) hypothetical protein (zm- |
| | | | | HPLV9) gene, a predicted Zea mays |
| | | | | calmodulin 5 gene (Phytozome gene ID |
| | | | | Zm00008a029682, WO Patent 2016109157) |
| | 26,708-26,716 | Intervening Sequence | 9 | DNA sequence used for cloning |
| | 26,717-28,156 | ipd079Ea | 1440 | Insecticidal protein gene from Ophioglossum |
| | | | | pendulum (WO Patent 2017023486) |
| | 28,157-28,173 | Intervening Sequence | 17 | DNA sequence used for cloning |
| | 28,174-29,126 | sb-SCI-1B Terminator | 953 | Terminator region of the Sorghum bicolor |
| | | | | (sorghum) subtilisin-chymotrypsin inhibitor 1B |
| | | | | gene (WO Patent 2018102131) |
| | 29,127-29,172 | Intervening Sequence | 46 | DNA sequence used for cloning |
| | 29,173-29,632 | Z27G Terminator | 460 | Terminator region from the Zea mays W64 line |
| | | | | 27-kDa gamma zein gene (Das et al., 1991; Liu |
| | | | | et al., 2016) |

### Example 2. Transformation of Maize by Agrobacterium Transformation and Regeneration of Transgenic Plants Containing the IPD079, PAT, and PMI Genes

DP-915635-4 maize event was produced by Agrobacterium-mediated SSI transformation with plasmid PHP83175. Agrobacterium-mediated SSI was essentially performed as described in U.S. patent application publication number 2017/0240911, herein incorporated by reference.

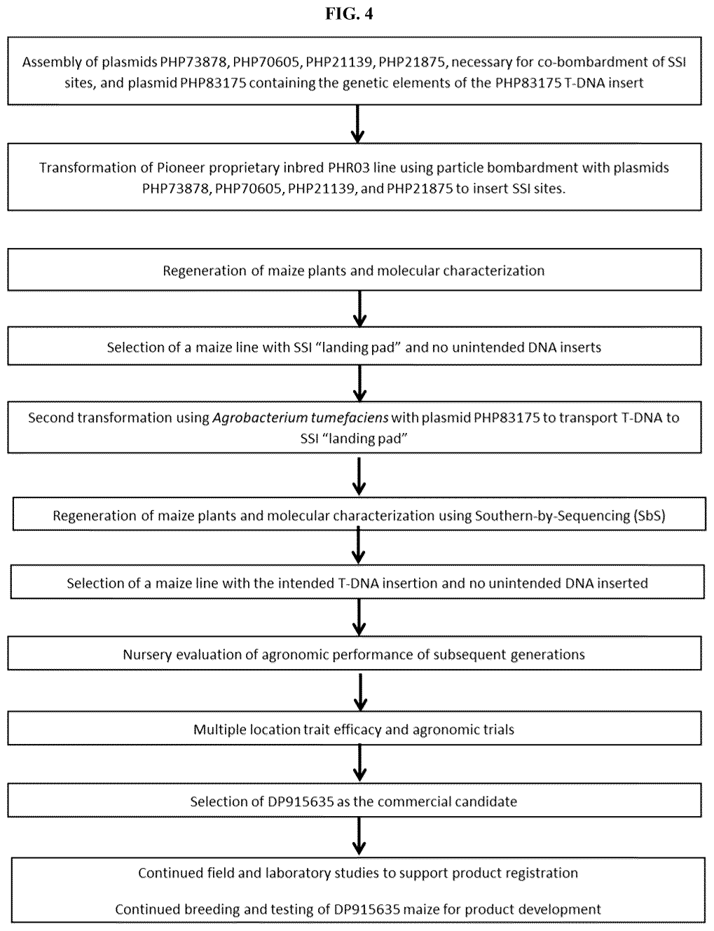

Over 2700 immature embryos were infected with PHP83175. After the 105-day selection and regeneration process, a total of 46 TO plantlets were regenerated. Samples were taken from all TO plantlets for PCR analysis to verify the presence and copy number of the inserted IPD079, PMI, and mo-PAT. In addition to this analysis, the TO plantlets were analyzed by PCR for the presence of certain Agrobacterium binary vector backbone sequences and for the developmental genes, zm-odp2 and zm-wus2 disclosed in U.S. Pat. Nos. 7,579,529 and 7,256,322, herein incorporated by reference in their entireties. Plants that were determined to contain single copy of the inserted genes, no Agrobacterium backbone sequences, and no developmental genes were selected for further greenhouse propagation. Samples from those PCR selected TO quality events were collected for further analysis using Southern-by-Sequencing to confirm that the inserted genes were in the correct target locus without any gene disruptions. Maize events DP-915635-4 were confirmed to contain a single copy of the T-DNA (See Examples 3 and 4). These selected TO plants were assayed for trait efficacy and protein expression. TO plants meeting all criteria were advanced and crossed to inbred lines to produce seed for further testing. A schematic overview of the transformation and event development is presented in FIG. 4.

### Example 3. Identification of Maize Events DP-915635-4

Genomic DNA from leaf tissue representing multiple generations of maize event DP-915635-4, known copy number calibrator controls, a negative control source (DNA from a non-genetically modified maize) and no template controls (NTC) were isolated and subjected to quantitative real-time PCR (qPCR) amplification using event-specific and construct-specific primer and probes. Real-time PCR analyses of DP-915635-4 maize DNA using event-specific and construct-specific assays confirm the stable integration and segregation of a single copy of the T-DNA of plasmid PHP83175 in leaf samples tested, as demonstrated by the quantified detection of event DP-915635-4, and ipd079Ea, pmi, and mo-PAT transgenes in DP-915635-4 maize. The reliability of each event-specific and construct-specific PCR method was assessed by repeating the experiment in quadruplicate. The sensitivity, or Limit of Detection (LOD) of the PCR amplification was evaluated by testing of various dilutions of the genomic DNA from DP-915635-4.

Two generations of maize containing event DP-915635-4 were grown in cell-divided flats under typical greenhouse production conditions. Approximately 100 seed were planted for each generation.

Leaf samples were collected from each healthy plant, when plants were between the

V5 and V9 growth stages. The samples were taken from the youngest leaf that was emerged from the whorl of each plant. Three leaf punches per plant were analyzed for the copy number of each event's genomic junction and the PHP83175 T-DNA through copy number PCR (qPCR) for the DP-915635-4event as well as ipd079Ea, pmi, and mo-PAT transgenes from seed grown at Pioneer Hi-Bred International, Inc. (Johnston, IA). Genomic DNA extractions from the leaf samples were performed using a high alkaline extraction protocol. Validated laboratory controls (copy number calibrators and negative) were prepared from leaf tissue using a standard cetyl trimethylammonium bromide (CTAB) extraction protocol.

Genomic DNA supporting laboratory controls were quantified using Quant-iT PicoGreen® reagent (Invitrogen, Carlsbad, CA). Quantification of genomic test and control samples were estimated using the NanoDrop 2000c Spectrophotometer using NanoDrop 2000/2000c V1.6.198 Software (ThermoScientific, Wilmington, DE).

Genomic DNA samples isolated from leaf tissue of DP-915635-4 as well as control samples were subjected to real-time PCR amplification utilizing event-specific and construct specific primers and probes which span specific regions of the PHP83175 T-DNA as well as the genomic junctions that span each insertion site for events DP-915635-4. An endogenous reference gene, High Mobility Group A (hmg-A) (Krech, et al. (1999). Gene 234: (1) 45-50) was used in duplex with each assay for both qualitative and quantitative assessment of each assay and to demonstrate the presence of sufficient quality and quantity of DNA within the PCR reaction. The PCR target sites and size of expected PCR products for each primer/probe set are shown in Table 2. Primer and probe sequence information supporting each targeted region are shown in Table 3. PCR reagents and reaction conditions are shown in Table 4. In this study approximately 3-ng of maize genomic DNA was used for all PCR reactions.

TABLE 2
PCR Genomic DNA Target Site and
Expected Size of PCR Products

| | | Expected | |
|---|---|---|---|
| | | Size of | |
| | | PCR | Amplicon |
| Primer and Probe | Targeted | Product | SEQ |
| Set | Regions | (bp) | ID NO: |
| SEQ ID NOs: 6-8 | DP-915635-4 | 72 | 21 |
| | insertion | | |
| SEQ ID NOs: 9-11 | ipd079Ea | 57 | 22 |
| SEQ ID NOs: 12-14 | pmi | 113 | 23 |
| SEQ ID NOs: 15-17 | mo-PAT | 76 | 24 |
| SEQ ID NOs: 18-20 | hmg-A | 79 | 25 |

TABLE 3
Primers and Probe Sequence and Amplicon for
PCR Genomic DNA Targeted Regions

| Reagent | Sequence (5′ to 3′) | Length (base) |
|---|---|---|
| SEQ ID NO: 6 | GCATCTAGGACCGACTAGCTAACTAAC | 27 |
| forward primer | | |
| SEQ ID NO: 7 | CTTTGCATCATGTCTTGAACAATG | 24 |
| reverse primer | | |
| SEQ ID NO: 8 | 6-FAM-CGCCATGAGGAGCAA-MGB | 15 |
| probe | | |
| SEQ ID NO: 9 | GCTGGCCGTGAAGGTGAA | 18 |
| forward primer | | |
| SEQ ID NO: 10 | TCCACGCTAGCGCTGAAGTA | 20 |
| reverse primer | | |
| SEQ ID NO: 11 | 6-FAM-CTCAGCGGAAGCTA-MGB | 14 |
| probe | | |
| SEQ ID NO: 12 | TGACTGTCAAAGGCCACGG | 19 |
| forward primer | | |
| SEQ ID NO: 13 | AGATGGACAAGTCTAGGTTCCACC | 24 |
| reverse primer | | |
| SEQ ID NO: 14 | 6-FAM-CCGTTTAGCGCGTGTTTACAACAAGCTG-BHQ | 28 |
| probe | | |
| SEQ ID NO: 15 | CATCGTGAACCACTACATCGAGAC | 24 |
| forward primer | | |
| SEQ ID NO: 16 | GTCGATCCACTCCTGCGG | 18 |
| reverse primer | | |
| SEQ ID NO: 17 | 6′FAM-ACCGTGAACTTCCGCACCGAGC-BHQ1 | 22 |
| probe | | |
| SEQ ID NO: 18 | TTGGACTAGAAATCTCGTGCTGA | 23 |
| forward primer | | |
| SEQ ID NO: 19 | GCTACATAGGGAGCCTTGTCCT | 22 |
| reverse primer | | |
| SEQ ID NO: 20 | VIC-GCGTTTGTGTGGATTG-MGB | 16 |
| probe | | |

SEQ ID NO: 21: DP-915635-4 assay amplicon sequence (72-bp; primer
and probe binding sites are in bold and underlined)
GCATCTAGGACCGACTAGCTAACTAACTAGGGCGCCATGAGGAGCAATCATTGTTCAAGACATGATGC
AAG
SEQ ID NO: 22: ipd079Ea assay amplicon sequence (57-bp; primer and
probe binding sites are in bold and underlined)
GCTGGCCGTGAAGGTGAAGCTCAGCGGAAGCTACGGCTACTTCAGCGCTAGCGTGGA
SEQ ID NO: 23: pmi assay amplicon sequence (113-bp; primer and probe
binding sites are in bold and underlined
TGACTGTCAAAGGCCACGGCCGTTTAGCGCGTGTTTACAACAAGCTGTAAGAGCTTACTGAAAAAATT
AACATCTCTTGCTAAGCTGGGGGTGGAACCTAGACTTGTCCATCT
SEQ ID NO: 24: mo-pat assay amplicon sequence (76-bp; primer and
probe binding sites are in bold and underlined)
CATCGTGAACCACTACATCGAGACCTCCACCGTGAACTTCCGCACCGAGCCGCAGACCCCGCAGGAGT
GGATCGAC
SEQ ID NO: 25: hmg-A assay amplicon sequence (79-bp; primer and
probe binding sites are in bold and underlined)
TTGGACTAGAAATCTCGTGCTGATTAATTGTTTTACGCGTGCGTTTGTGTGGATTGTAGGACAAGGCT
CCCTATGTAGC

TABLE 4
PCR Reagents and Reaction Conditions

| | | | Temperature | Time | |
|---|---|---|---|---|---|
| Step | Description | | (° C.) | (seconds) | Cycles |
| 1 | Initial Denaturation | | 95 | 120 | 1 |
| 2a | Amplification | Denaturation | 95 | 1 | 40^{a} |
| 2b | | Anneal/Extend | 60 | 20 | |

^{a}If thermal cycling is completed using a Roche LightCycler ® 480, initial
denaturation was 300 seconds for step 1 with 45 cycles for steps 2a and 2b.

PCR products ranging in size from 57-bp to 113-bp, representing the insertion sites for event DP-915635-4 as well as the transgenes within the T-DNA from plasmid PHP83175, were amplified and observed in 100 individual leaf samples from event DP-915635-4 as well as eight copy number calibrator genomic controls, but were absent in each of the eight negative genomic controls and eight NTC controls. Each assay was performed a total of four times with the same results observed. Cr values were calculated for each sample and all positive controls.

Using the maize endogenous reference gene hmg-A, a PCR product of 79-bp was amplified and observed in 100 individual leaf samples each from event DP-915635-4 as well as eight copy number calibrator and eight negative genomic controls. Amplification of the endogenous gene was not observed in the eight No Template (NTC) controls tested with no generation of Cr values. For each sample, each assay was performed in duplex with both insertion sites and all transgenes a total of four times with the same results observed each time. Cr values were calculated for each sample and all positive and negative controls.

To assess the sensitivity of the construct-specific PCR assays, DP-915635-4 maize DNA was diluted in control maize genomic DNA, resulting in test samples containing various amounts of event DP-915635-4 DNA (5-ng, 1-ng, 500-pg, 250 μg, 100-pg, 50-pg, 20-pg, 10-pg, 5-pg) in a total of 5-ng maize DNA. These various amounts of DP-915635-4 maize DNA correspond to 100%, 20%, 10%, 5%, 2%, 1%, 0.4%, 0.2%, and 0.1% of DP-915635-4 maize maize DNA in total maize genomic DNA, respectively. The various amounts of DP-915635-4 DNA were subjected to real-time PCR amplification for transgenes ip079Ea, PMI, and mo-PAT. Based on these analyses, the limit of detection (LOD) in 5-ng of total DNA for event DP-915635-4 was determined to be approximately 20-pg for ip079Ea, or 0.4%, 250-pg for pmi, or 5%, and 5-pg for mo-pat, or 0.1% (DP-915635-4). The determined sensitivity of each assay described is sufficient for many screening applications. Each concentration was tested a total of four times with the same results observed each time.

Real-time PCR analyses of event DP-915635-4 utilizing event-specific and construct-specific primer/probe sets for event DP-915635-4 confirm the stable integration and segregation of a single copy of the T-DNA of plasmid PHP83175 of the event in leaf samples tested, as demonstrated by the quantified detection of ipd079Ea, pmi, and mo-PAT transgenes in DP-915635-4 maize. These results were reproducible among all the replicate qPCR analyses conducted. The maize endogenous reference gene assay for detection of hmg-A amplified as expected in all the test samples, negative controls and was not detected in the NTC samples. The sensitivity of each assay under the conditions described ranges from 5-pg to 250-pg DNA, all sufficient for many screening applications by PCR.

### Example 4. Southern-by-Sequencing (SbS) Analysis of DP-915635-4 Maize for Integrity And Copy Number

Southern-by-Sequencing (SbS) utilizes probe-based sequence capture, Next Generation Sequencing (NGS) techniques, and bioinformatics procedures to isolate, sequence, and identify inserted DNA within the maize genome. By compiling a large number of unique sequencing reads and comparing them to the transformation plasmid, unique junctions due to inserted DNA are identified in the bioinformatics analysis and can be used to determine the number of insertions within the plant genome. The TO plant of DP-915635-4 maize was analyzed by SbS to determine the insertion copy number. In addition, samples of the control maize line were analyzed.

Genomic DNA was extracted from the TO generation of DP-915635-4 maize and control plants.

Capture probes used to select PHP83175 plasmid sequences were designed and synthesized by Roche NimbleGen, Inc. (Madison, WI). A series of unique sequences encompassing the plasmid sequence was used to design overlapping biotinylated oligonucleotides as capture probes. The probe set was designed to target most sequences within the PHP83175 transformation plasmid during the enrichment process. The probes were compared to the maize genome to determine the level of maize genomic sequence that would be captured and sequenced simultaneously with the PHP83175 plasmid sequence.

Next-generation sequencing libraries were constructed for the DP-915635-4 maize plants and the control maize lines. SbS was performed as described by Zastrow-Hayes, et al. Plant Genome (2015). The sequencing libraries were hybridized to the capture probes through two rounds of hybridization to enrich the targeted sequences. Following NGS on a HiSeq 2500 (Illumina, San Diego, CA), the sequencing reads were assessed for trimming and quality assurance. Reads were aligned against the maize genome and the transformation construct and reads that contain both genomic and plasmid sequence were identified as junction reads. Alignment of the junction reads to the transformation construct shows borders of the inserted DNA relative to the expected insertion.

To identify junctions that included endogenous maize sequences, control maize genomic DNA libraries were captured and sequenced in the same manner as the DP-915635-4 maize plants. These libraries were sequenced to an average depth approximately five times that of the depth for the DP-915635-4 maize plant samples. This increased the probability that the endogenous junctions captured by the PHP83175 probes would be detected in the control samples, so that they could be identified and removed in the DP-915635-4 maize samples.

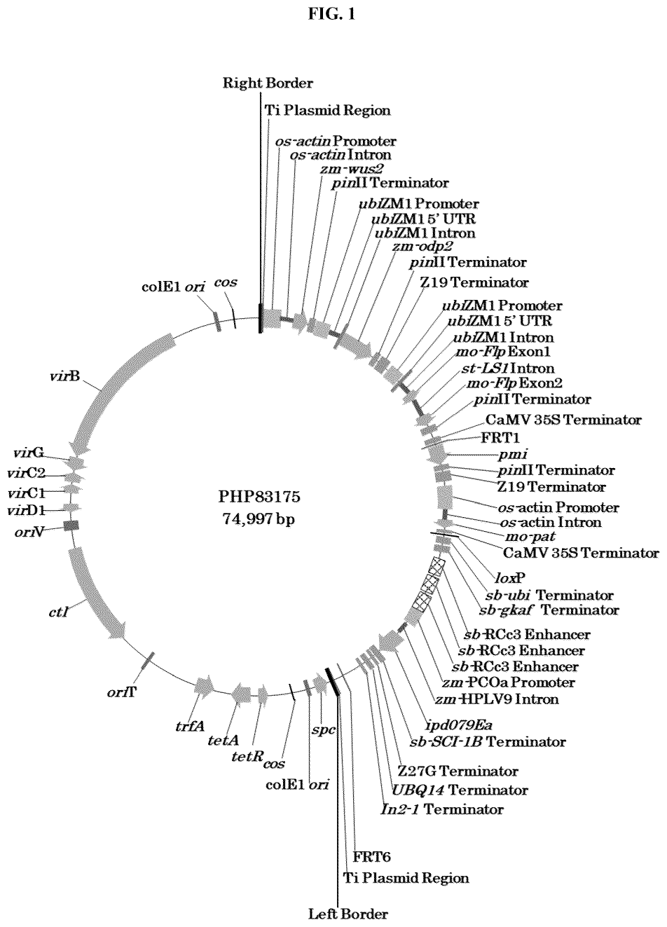

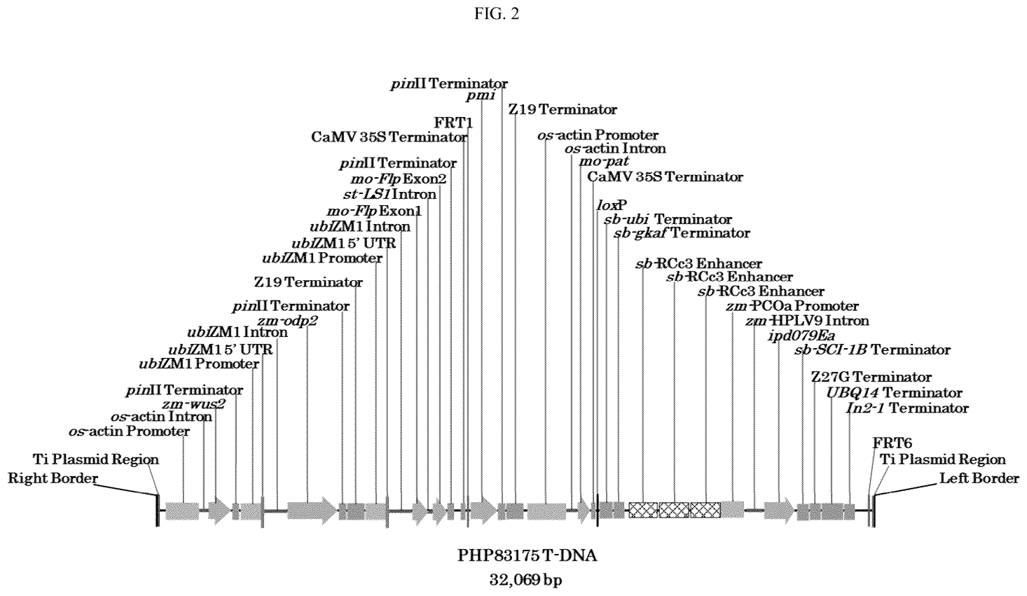

Integration and copy number of the insertion were determined in DP-915635-4 maize derived from construct PHP83175. Schematic maps of the PHP83175 plasmid and the T-DNA from PHP83175 used in transformation are provided in FIGS. 1 and 2.

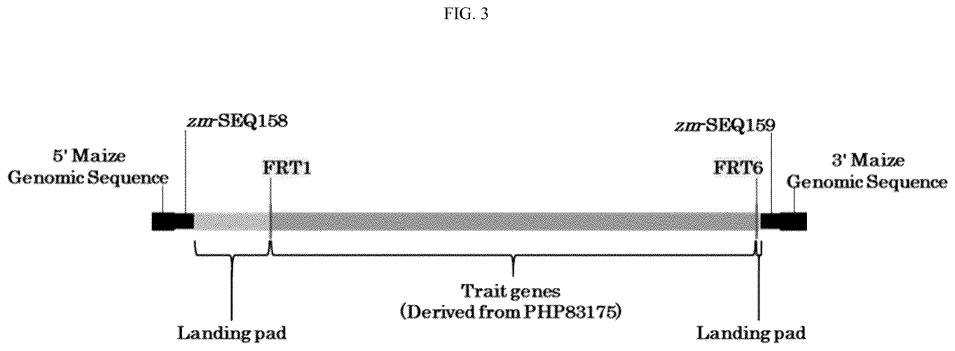

SbS was conducted on the TO plant of DP-915635-4 maize to determine the insertion copy number in the genome. Alignment of the SbS reads to the expected insertion region (including the landing pad elements zm-SEQ158 and zm-SEQ159; FIG. 3) resulted in two unique junctions between the genomic flanking sequence and the landing pad. The FRT1 and FRT6 sites are the two locations where the target trait genes from PHP83175 were integrated into the site-specific integration (SSI) landing pad. There were no other junctions between the PHP83175 sequences and the maize genome detected in the plant, indicating that there are no additional plasmid-derived insertions present in DP-915635-4 maize. Additionally, there were no junctions between non-contiguous regions of the PHP83175 T-DNA identified, indicating that there are no detectable rearrangements or truncations in the inserted DNA. Furthermore, there were no junctions between maize genome sequences and the backbone sequence of PHP83175 in the plant analyzed, demonstrating that no plasmid backbone sequences were incorporated into DP-915635-4 maize.

SbS analysis of the TO plant of DP-915635-4 maize demonstrated that there is a single insertion containing the desired genes from the PHP83175 T-DNA in DP-915635-4 maize and that no additional insertions are present in the respective genomes.

Southern-by-Sequencing (SbS) analysis was conducted on the TO plant of DP-915635-4 maize to confirm insertion copy number. The results indicate a single PHP83175 T-DNA insertion in the plant. No junctions between the PHP83175 T-DNA sequences and the maize genome were detected in control plants, indicating that, as expected, these plants did not contain any insertions derived from PHP83175. Furthermore, no plasmid backbone sequences were detected in the plant analyzed. SbS analysis of the TO plant of DP-915635-4 maize demonstrated that there is a single insertion of the PHP83175 T-DNA in DP-915635-4 maize and that no additional insertions are present in the respective genomes. A single nucleotide change, A to C change at bp 2931 in the ubiZMI promoter of the complete insert and flanking region sequence as shown in SEQ ID NO: 32, was identified in all five plants in the ubiZMI promoter of the pmi cassette that differs from the expected insertion sequence. As this change is in all five positive plants, it was determined to be present in the initial transformed plant. An additional single nucleotide change of a G to an A at position 8199 in SEQ ID NO: 32 was identified in the os-actin promoter of one plant of five plants; as this is the only occurrence it is likely due to a spontaneous change during the breeding process. Alignments of the reads from the five positive plants to the five plasmid maps show coverage of the genetic elements found in the intended insertion, along with coverage of the endogenous elements in the plasmids that were not incorporated into the insertion (zm-SEQ158, zm-SEQ159, zm-U6 pol III CHR8 promoter and terminator, zm-45CR1 guide RNA, In2-2 promoter, zm-wus2, and zm-odp2). Reads also aligned to the pinII terminator elements located outside of the intended insertion regions in PHP83175, PHP73878, PHP70605, and PHP21875 although these elements were not incorporated into the insertion. The NGS reads that aligned to these copies of the pinII terminator are from fragments containing the pinII terminator in the pmi cassette of the intended insertion; however, the reads from this single copy align to all copies of the pinII terminator in the plasmid maps. Similarly, reads aligned to the CaMV 35S terminator elements in the mo-Flp cassette and to the os-actin promoter and intron region of the zm-wus2 cassette in PHP83175 due to the presence of identical elements in the mo-pat cassette of the intended insertion.

### Example 5. Insect Efficacy of Maize Events DP-915635-4

μl hybrid maize lines containing the insect-active IPD079Ea protein were evaluated in the field for protection against corn rootworms (CRW) by testing multiple events including DP-915635-4 maize. Data were statistically analyzed using a linear mixed model. Field testing was conducted in 13 locations located in commercial maize-growing regions of North America: Brookings, SD (BR); Mankato, MN (MK); Marion, IA (MR); Readlyn, IA (MR_RE); Johnston, IA (JH_D2); Johnston, IA (JH_D3); Gilbert, IA (JH_GB); Watertown, WI (JV); Shabonna, IL (JV_SH); Seymour, IL (CI_SE); Fowler, IN (WN); York, NE (YK); and Lindsey, NE (YK_LI). No efficacy data were collected at six of the 13 locations (sites JH_GB, JV, JV_SH, CI_SE, YK, & YK_LI) due to a low nodal injury score (CRWNIS) below 0.75 on negative control roots.

Single-row plots (10 feet in length) were planted in a randomized complete block experimental design with two replications. Prior to planting, 168 kernels from each seed lot were characterized by PCR analysis to confirm the presence of the traits. A four-foot length of each row was manually infested utilizing a tractor-mounted CRW egg infester at a targeted infestation rate of approximately 750 eggs/plant or 1500 eggs/plant, depending on the location, when plants reached the V2-V4 growth stages. Eggs were injected into the soil approximately 4 inches deep and approximately 2-3 inches on both sides of each plant. Injury from larval feeding on roots was evaluated between 56 and 71 days after planting. Two corn roots were tagged, manually dug from the ground, washed clean of soil with pressurized water, and evaluated for the amount of larval feeding at approximately the R2 growth stage. Root injury was evaluated by visually rating and recording the amount of larval feeding contained on each root using the Iowa State 0-3 node-injury scale.

The mean node-injury root rating results from CRW for both DP-915635-4 maize and control maize are provided in Table 5. These results indicate that maize lines containing the insect-active IPD079Ea protein are efficacious against CRW.

TABLE 5
Efficacy Results Against Corn Rootworm

| | Number | Mean Node- | | |
|---|---|---|---|---|
| Maize | of | Injury Root | | |
| Line | Plots | Rating ± SD | Range | P-Value |
| DP915635 | 14 | 0.16 ± 0.17 | 0.02-0.71 | <0.0001^{a} |
| Control | 14 | 2.02 ± 0.80 | 0.70-3.00 | |

^{a}Statistically significant difference; (P-value < 0.05)

TABLE 6
Efficacy Results Against Corn Rootworm

| | Number | Mean Node- | | |
|---|---|---|---|---|
| Maize | of | Injury Root | | |
| Line | Plots | Rate ± SD | Range | P-Value |
| DP-915635-4 | 27 | 0.13 ± 0.08 | 0.02-0.70 | <0.0001^{a} |
| Control | 27 | 1.79 ± 0.74 | 0.50-3.00 | |

^{a}Statistically significant difference; (P-value < 0.05)

### Example 6. Agronomic and Yield Field Evaluations of Maize Events DP-915635-4

Agronomic field trials containing DP-915635-4 were to generate yield data and to evaluate other agronomic characteristics. All inbred and hybrid materials tested for an event were generated from a single TO plant.

### Hybrid Trials

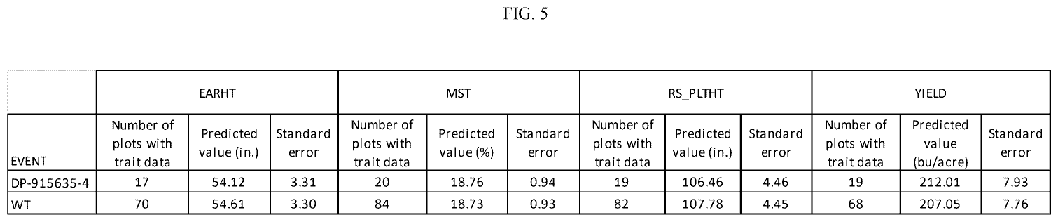

Hybrid trials were planted at 16 locations with a single replicate of the entry list at each location. Grain was harvested from 12 of the 16 locations. Each entry in a common background was crossed to three testers to generate hybrid seed for testing. Experiments were nested by testers, with the entries randomized within each nest. Various observations and data were collected at each planted location throughout the growing season. The following agronomic characteristics were analyzed for comparison to a wild type entry (WT), or an entry with the same genetics but without IPD079Ea, also referred to as base comparator (Table 7 and FIG. 5):
1.) Ear height (EARHT): Measurement from the ground to the attachment point of the highest developed ear on the plant. Ear height is measured in inches.
2.) Plant height (PLTHT): Measurement by drones from the ground to the base of the flag leaf. Plant height is measured in inches.
3.) Moisture (MST): Measurement of the percent grain moisture at harvest.
4.) Yield: Recorded weight of grain harvested from each plot. Calculations of reported bu/acre yields were made by adjusting to measured moisture of each plot.

### Inbred Trials

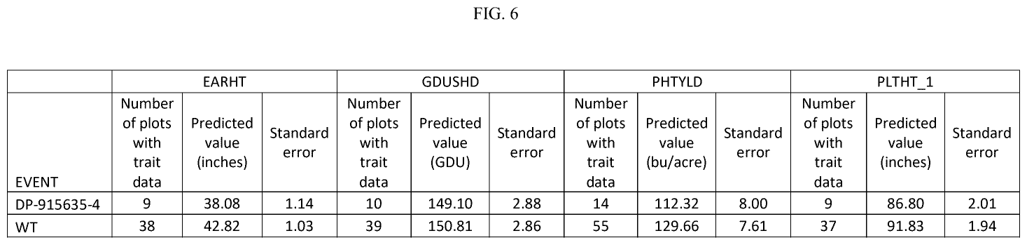

Inbred trials were planted at 8 locations with 2 replicates of the entry list at each location. Grain was harvested from 7 locations for analysis. One replicate at each location was nested by construct design; the other replicate was planted as a randomized complete block. Agronomic data and observations were collected for the inbred trials and analyzed for comparison to a wild type entry (WT), or untraited version of the same genotype. Data generated for the inbred trials included the following agronomic traits (Table 8 and FIG. 6):
1.) Growing degree units to shed (GDUSHD): Measurement records the total accumulated growing degree units when 50% of the plants in the plot have tassels that are shedding pollen. A single day equivalent is approximately 2.5 growing degrees units for this data set.
2.) Ear height (EARHT): Measurement from the ground to the attachment point of the highest developed ear on the plant. Ear height is measured in inches.
3.) Plant height (PLTHT): Measurement from the ground to the base of the flag leaf. Plant height is measured in inches.
4.) Ear photometry yield (PHTYLD): Calculated yield estimates from images of harvested ears from each plot. Units for the values shown are bu/acre.

### Trial Results

To evaluate the hybrid data, a mixed model framework was used to perform multi location analysis. In the multi-location analysis, main effect construct design is considered as fixed effect. Factors for location, background, tester, event, background by construct design, tester by construct design, tester by event, location by background, location by construct design, location by tester, location by background by construct design, location by tester by construct design, location by event, location by tester by event are considered as random effects. The spatial effects including range and plot within locations were considered as random effects to remove the extraneous spatial noise. The heterogeneous residual was assumed with autoregressive correlation as AR1*AR1 for each location. The estimate of construct design and prediction of event for each background were generated. The T-tests were conducted to compare construct design/event with WT. A difference was considered statistically significant if the P-value of the difference was less than 0.05. Yield analysis was by ASREML (VSN International Ltd; Best Linear Unbiased Prediction; Cullis, B. Ret al (1998) Biometrics 54:1-18, Gilmour, A. R. et al (2009); ASReml User Guide 3.0, Gilmour, A. R., et al (1995) Biometrics 51:1440-50).

To evaluate the inbred data, a mixed model framework was used to perform multi location analysis. In the multi-location analysis, main effect construct design is considered as fixed effect. Factors for location, background, event, background by construct design, location by background, location by construct design, location by background by construct design, location by event and rep within location are considered as random effects. The spatial effects including range and plot within locations were considered as random effects to remove the extraneous spatial noise. The heterogeneous residual was assumed with autoregressive correlation as AR1*AR1 for each location. The estimate of construct design and prediction of event for each background were generated. The T-tests were conducted to compare construct design/event with WT. A difference was considered statistically significant if the P-value of the difference was less than 0.05. Yield analysis was by ASREML (VSN International Ltd; Best Linear Unbiased Prediction; Cullis, B. Ret al (1998) Biometrics 54:1-18, Gilmour, A. R. et al (2009); ASReml User Guide 3.0, Gilmour, A. R., et al (1995) Biometrics 51:1440-50).

TABLE 7
Hybrid performance of events DP-915635-4
compared to base entry-yield

| | Number | | | | |
|---|---|---|---|---|---|
| | of plots | Predicted | | Predicted | Predicted |
| | with yield | value | Standard | lower | upper 95% |
| Event | data | (bu/acre) | Error | 95% CL | CL |
| WT (base | 68 | 207.05 | 7.76 | 191.28 | 222.83 |
| comparator) | | | | | |
| DP-915635-4 | 19 | 212.01 | 7.93 | 195.87 | 228.15 |

TABLE 8
Inbred performance of events DP-915635-4
compared to base entry-yield

| | Number | | | | |
|---|---|---|---|---|---|
| | of plots | Predicted | | Predicted | Predicted |
| | with yield | value | Standard | lower | upper 95% |
| Event | data | (bu/acre) | Error | 95% CL | CL |
| DP-915635-4 | 14 | 112.32 | 8.00 | 96.04 | 128.59 |
| WT (base | 55 | 129.66 | 7.61 | 114.18 | 145.14 |
| comparator) | | | | | |

### Example 7. Protein Expression and Concentration

### Protein Extraction

For analysis of IPD079Ea protein concentrations, processed root tissue sub-samples were weighed at a target weight of 20 mg. For analysis of PAT and PMI protein concentrations, processed leaf tissue sub-samples were weighed at a target weight of 10 mg. Samples were extracted with 0.60 ml of chilled phosphate-buffered saline containing polysorbate 20 (PBST). Extracted samples were centrifuged, and then supernatants were removed and prepared for analysis.

### Determination of IPD079Ea Protein Concentration

Prior to analysis, samples were diluted as applicable in PBST. Standards (typically analyzed in triplicate wells) and diluted samples (typically analyzed in duplicate wells) were incubated in a plate pre-coated with an IPD079Ea-specific antibody. Following incubation, unbound substances were washed from the plate and the bound IPDO7Ea protein was incubated with a different IPD079Ea-specific antibody conjugated to the enzyme horseradish peroxidase (HRP). Unbound substances were washed from the plate. Detection of the bound IPD079Ea-antibody complex was accomplished by the addition of substrate, which generated a colored product in the presence of HRP. The reaction was stopped with an acid solution and the optical density (OD) of each well was determined using a plate reader.

### Determination of PAT Protein Concentration

Prior to analysis, samples were diluted as applicable in PBST. Standards (typically analyzed in triplicate wells) and diluted samples (typically analyzed in duplicate wells) were co-incubated with a PAT-specific antibody conjugated to the enzyme HRP in a plate pre-coated with a different PAT-specific antibody. Following incubation, unbound substances were washed from the plate. Detection of the bound PAT-antibody complex was accomplished by the addition of substrate, which generated a colored product in the presence of HRP. The reaction was stopped with an acid solution and the OD of each well was determined using a plate reader.

### Determination of PMI Protein Concentration

Prior to analysis, samples were diluted as applicable in PBST. Standards (typically analyzed in triplicate wells) and diluted samples (typically analyzed in duplicate wells) were incubated in a plate pre-coated with a PMI-specific antibody. Following incubation, unbound substances were washed from the plate and the bound PMI protein was incubated with a different PMI-specific antibody conjugated to the enzyme HRP. Unbound substances were washed from the plate. Detection of the bound PMI-antibody complex was accomplished by the addition of substrate, which generated a colored product in the presence of HRP. The reaction was stopped with an acid solution and the OD of each well was determined using a plate reader.

### Calculations for Determining Protein Concentrations

SoftMax Pro GxP (Molecular Devices) microplate data software was used to perform the calculations required to convert the OD values obtained for each set of sample wells to a protein concentration value.

A standard curve was included on each ELISA plate. The equation for the standard curve was derived by the software, which used a quadratic fit to relate the OD values obtained for each set of standard wells to the respective standard concentration (ng/ml).

$$Adjusted Concentration = Interpolated Sample Concentration \times Dilution Factor$$

Adjusted sample concentration values obtained from SoftMax Pro GxP software were converted from ng/ml to ng/mg sample weight as follows:

$$Sample Concentration ( ng protein / mg sample weight )$$

$$Sample Concentration ( ng / ml ) \frac{Extraction Buffer Volume ( ml )}{Sample Target Weight ( mg )}$$

### Results

Protein concentration results (means, standard deviations, and ranges) were determined for IPD079Ea protein in V9 root tissue and the PAT and PMI proteins in V9 leaf tissue from two generations of DP-915635-4 maize.

TABLE 9
Expressed Trait Protein Concentration Results

| | | | | | | Number of Samples |
|---|---|---|---|---|---|---|
| | Tissue | | Protein Concentration | | | <LLOQ/ |
| | (Growth | | (ng/mg Tissue Dry Weight) | | | Total Number of |
| Protein | Stage) | Generation | Mean ± SD | Range | Sample LLOQ | Samples Reported |
| IPD079Ea | Root (V9) | T3 | 22 ± 7.0 | 14-29 | 0.069 | 0/5 |
| | | F1 | 20 ± 3.1 | 15-23 | 0.069 | 0/5 |
| PAT | Leaf (V9) | T3 | 6.3 ± 0.64 | 5.6-7.2 | 0.11 | 0/5 |
| | | F1 | 5.1 ± 0.63 | 4.2-5.9 | 0.11 | 0/5 |
| PMI | Leaf (V9) | T3 | 11 ± 2.5 | 7.8-14 | 0.54 | 0/5 |
| | | F1 | 5.8 ± 0.86 | 4.9-7.2 | 0.54 | 0/5 |

The above description of various illustrated embodiments of the disclosure is not intended to be exhaustive or to limit the scope to the precise form disclosed. While specific embodiments of and examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the scope to the specific embodiments disclosed in the specification and the claims.

The entire disclosure of each document cited (including patents, patent applications, journal articles, abstracts, manuals, books or other disclosures) in the Background, Detailed Description, and Examples is herein incorporated by reference in their entireties.

Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees celsius; and pressure is at or near atmospheric.

1-53. (canceled)

54. A corn plant expressing an insecticidal protein from a fern and exhibiting a mean node-injury root rating of at least about 0.15 against corn root worm in combination with a bacterial protein exhibiting insecticidal activity against corn root worm and optionally expressing a dsRNA that targets an endogenous gene of corn rootworm, wherein the insecticidal protein, the bacterial insecticidal protein and the dsRNA display different mode of actions.

55. A corn plant expressing three distinct modes of actions against western corn rootworm, wherein at least two modes are due to insecticidal proteins not derived from a bacteria.
